SEQUENCE LISTING (6 sequences)

SEQ ID NO: 1 - DNA, 14588 bp, Artificial
  Clostridium/E. coli shuttle vector pGn15_SUC(op)
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgcgccattcgccattcaggctgcgcaactgttgggaagggcgatcggtgcgggcctcttcgctattacgccagtctagacagggccccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgatcgggccccctaggcctctggcgaaagggggatgtgctgcaaggcgattaagttgggtaacgccagggttttcccagtcacgacgttgtaaaacgacggccagtgccaagcttgagcttaatcgcactggccggccgcttataatccataacaatcatcctttctgtgacactgtcagacacttatcacattaagtatatactattattaaactattctatatacttaatttattttaatagaaaaacataatatcataataacttcaaaattaaactttatttatgatttcatacttgactttgattttagaaaggatatactttttagcagatttggaaacggctttggacgtagtttgcccatagatgaacaaacaaactacatccaaaaattatacttttcccttcattggtatccgtatttttacatcttaatagcgtatgtattacaacacacctaaacaacgaccttacggtctgctactgcatatcctagcttgattgtttagttgcctcaactatgcttaaccctaccccgaactctttttttattgtgggttttcgtcgtgaagtcccaccgacacataatcataacataagatgtattatgaaaatgcgagtgactatccttttgtatcggctcactacaccacagatatattttttagtgcatactgtgtcggcactctcaatattaaattaataaataaattattttttctttttactcttctttacatgagcttttttaaagctccttgcataatatttaatgcatgtacgttcttttttctgttcttcctctgtaaaacatctcatttttatggcacaccatccatatcggttcatctttgaacaattaatacattggactttccctttatgtaaacatcttgactcattgtatttactgcaataggttgctactgttttatcatgaatcatcaacgaatgtattttgcatatttcagtatctttaataaactctttgcaatttttacaagttttcatatacgcccttcttttcataaattaatttatgaattctatgtattccaaggagctttttaaagctccccctttcgtactacttagctacaagcactataaaagtcataatgtttactgctaaagtcaataatgatattgctaataccttgttatttgataagatactgctttcctctgtcactttgctcaccccctttcattttcataaattaattatgaaaaaataaatatacttctaatttttatcaaataaaaaagcctttgcgtactgcttccaatacacaaaggctataaacttctaaatcttacttattgcaatttacatttatatctgttaagataatctcataaattgaatatatatagttaatgtttctcttgtatgtcggtacatttgaaatattgctatagatagagttctctaacggcttgatgtgttggtagcacatttaagttttggcttatatactaagttggtagcttaatatataagagctgaggacttatttttttattaaatttttcaacttgtctatattttaacccgtaattgaatacataacaagtatttttttttgtattcaattaaacattcataaatgagtataattaatcatactaaattctataattttcttttctgtaaatttctttctattcagcactgttatgccttttgactatcacttaataaaaaataagaaatgaattgtcaattgttcaaaaaaataatggcggcgcgccatgcaaaagacataatcgattcacaaaaaataggtacacgaaaaacaagttaagggatgcagtttatgcatcccttaacttacttattaaataatttatagctattgaaaagagataagaattgttcaaagctaatattgtttaaatcgtcaattcctgcatgttttaaggaattgttaaattgattttttgtaaatattttcttgtattctttgttaacccatttcataacgaaataattatacttctgtttatctttgtgtgatattcttgatttttttctatttaatctgataagtgagctattcactttaggtttaggatgaaaatattctcttggaaccatacttaatatagaaatatcaacttctgccattaaaaataatgccaatgagcgttttgtatttaataatcttttagcaaacccgtattccacgattaaataaatctcatcagctatactatcaaaaacaattttgcgtattatatccgtacttatgttataaggtatattaccaaatattttataggattggtttttaggaaatttaaactgcaatatatccttgtttaaaacttggaaattatcgtgatcaacaagtttattttctgtagttttgcataatttatggtctatttcaatggcagttacgaaattacacctctgtactaattcaagggtaaaatgcccttttcctgagccgatttcaaagatattatcatgttcatttaatcttatatttgtcattattttatctatattatgttttgaagtaataaagttttgactgtgttttatatttttctcgttcattataaccctctttattttttcctccttataaaattagtataattatagcacgagctctgataaatatgaacatgatgagtgatcgttaaatttatactgcaatctgatgcgattattgaataaaagatatgagagatttatctagtttctttttttacaagaaaaaagaaagttcttaaaggttttatacttttggtcgtagagcacacggtttaacgacttaattacgaagtaaataagtctagtgtgttagactttaatgtttttttaaggcattagtgcatttaagcgtcagagcatggctttatgccgagaaaactattggttggaatggcgtgtgtgttagccaaagctcctgcagtaactagtatcacaagtttgtacaaaaaagcaggcttaaggctcctaagtgaaatatatacatattataacaataaaataagtattagtgtaggatttttaaatagagtatctattttcagattaaatttttgcttatttgatttacattatataatattgagtaaagtattgactagcaaaattttttgatactttaatttgtgaaatttcttatcaaaagttatatttttgaatgatttttattgaaaaatacaactaaaaaggattatagtataagtgtgtgtaattttgtgttaaatttaaggcagcgttccaggagggacagaacaatgaacttacatgaatatcaggcaaaacaactttttgcccgctatggcttaccagcaccggtgggttatgcctgtactactccgcgcgaagcagaagaagccgcttcaaaaatcggtgccggtccgtgggtagtgaaatgtcaggttcacgctggtggccgcggtaaagcgggcggtgtgaaagttgtaaacagcaaagaagacatccgtgcttttgcagaaaactggctgggcaagcgtctggtaacgtatcaaacagatgccaatggccaaccggttaaccagattctggttgaagcagcgaccgatatcgctaaagagctgtatctcggtgccgttgttgaccgtagttcccgtcgtgtggtctttatggcctccaccgaaggcggcgtggaaatcgaaaaagtggcggaagaaactccgcacctgatccataaagttgcgcttgatccgctgactggcccgatgccgtatcagggacgcgagctggcgttcaaactgggtctggaaggtaaactggttcagcagttcaccaaaatcttcatgggcctggcgaccattttcctggagcgcgacctggcgttgatcgaaatcaacccgctggtcatcaccaaacagggcgatctgatttgcctcgacggcaaactgggcgctgacggcaacgcactgttccgccagcctgatctgcgcgaaatgcgtgaccagtcgcaggaagatccgcgtgaagcacaggctgcacagtgggaactgaactacgttgcgctggacggtaacatcggttgtatggttaacggcgcaggtctggcgatgggtacgatggacatcgttaaactgcacggcggcgaaccggctaacttccttgacgttggcggcggcgcaaccaaagaacgtgtaaccgaagcgttcaaaatcatcctctctgacgacaaagtgaaagccgttctggttaacatcttcggcggtatcgttcgttgcgacctgatcgctgacggtatcatcggcgcggtagcagaagtgggtgttaacgtaccggtcgtggtacgtctggaaggtaacaacgccgaactcggcgcgaagaaactggctgacagcggcctgaatattattgcagcaaaaggtctgacggatgcagctcagcaggttgttgccgcagtggaggggaaataacaaggcaaagtgaggaggtaaggtatgagtaatgaagtatctataaaagaattaattgaaaaggcaaaggtggcacaaaaaaaattggaagcctatagtcaagaacaagttgatgtactagtaaaagcactaggaaaagtggtttatgataatgcagaaatgtttgcaaaagaagcagttgaagaaacagaaatgggtgtttatgaagataaagtagctaaatgtcatttgaaatcaggagctatttggaatcatataaaagacaagaaaactgtaggcataataaaagaagaacctgaaagggcacttgtttatgttgctaagccaaagggagttgtggcagctactacgcctataactaatccagtggtaactcctatgtgtaatgcaatggctgctataaagggcagaaatacaataatagtagcaccacatcctaaagcaaagaaagtttcagctcatactgtagaacttatgaatgctgagcttaaaaaattgggagcaccagaaaatatcatacagatagtagaagcaccatcaagagaagctgctaaggaacttatggaaagtgctgatgtagttattgctacaggcggtgctggaagagttaaagctgcttactccagtggaagaccagcttatggcgttggacctggaaattcacaggtaatagttgataagggatacgattataacaaagctgcacaggatataataacaggaagaaaatatgacaatggaattatatgttcttcagagcaatcagttatagctcctgctgaagattatgataaggtaatagcagcttttgtagaaaatggggcattctatgtagaagatgaggaaacagtagaaaagtttagatcaactttatttaaagatggaaaaataaacagcaagattataggtaaatccgtccaaattattgcggatcttgcaggagtaaaagtaccagaaggtactaaggttatagtacttaagggtaaaggtgcaggagaaaaagatgtactttgtaaagaaaaaatgtgtccagttttagtagcattgaaatatgatacttttgaagaagcagttgaaatagctatggctaattatatgtatgaaggagctggtcatacagcaggcatacattctgacaatgacgagaacataagatatgcaggaactgtattacctataagcagattagttgtaaatcagcctgcaactactgctggaggaagtttcaataatggatttaaccctactactacactaggctgcggatcatggggcagaaacagtatttcagaaaatcttacttacgagcatcttataaatgtttcaagaatagggtatttcaataaagaagcaaaagttcctagctatgaggaaatatggggataaaagaggaggttaggcatgcagctgtttaaactcaagagtgtaacacatcatttcgatacttttgcagaatttgctaaagagttctgtttaggagaaagagatcttgtgatcactaacgaatttatctacgaaccatatatgaaagcttgtcaattaccttgtcactttgttatgcaggaaaagtatggtcaaggtgaacctagtgatgaaatgatgaacaatatactcgcagacattcgtaatattcagtttgatagagtcataggaataggaggaggtactgtgatagacatcagtaagctatttgttcttaaaggtctcaatgacgttttagatgcatttgatagaaaaatccccttaatcaaagagaaggaactcatcatagttcctacaacatgtggtacaggaagtgaagtaacgaatataagcatagctgaaatcaaatcaagacacactaaaatgggtttagctgatgatgcaatagtagcagatcatgcgattataatacccgaactacttaaatctctcccttttcatttttatgcttgtagtgctattgatgcattgattcatgcaattgagtcttatgtaagccctaaagcaagtccatatagcagattattcagtgaagcagcatgggatataatactcgaggtttttaaaaagattgctgaacatggaccagaatataggtttgagaagttaggagagatgataatggcttcgaattatgcaggtattgcatttggaaatgctggtgttggtgctgtacatgcattatcttaccctttaggtggaaattatcatgttccacatggagaagcaaactatcaattttttaccgaagtttttaaagtttatcaaaagaaaaatccgtttggatatatcgttgagttaaactggaaactttccaaaattctcaattgtcaacctgaatacgtatatcctaagctagatgaacttctcggttgtcttttgaccaaaaaacccttacatgaatatggaatgaaagatgaagaagttagaggatttgctgaatctgtgttaaagacgcaacaaagattattggctaacaattatgtcgaacttacagtagatgagatagaagggatttataggagactttactagagcttccgcggaggccggcgcggccgatttaaatcctcagcctgagcccccgggcggtatcagctcactcaaaggcggtaataacaactttgtatacaaaagttgtagtgtagtagcctgtgaaataagtaaggaaaaaaaagaagtaagtgttatatatgatgattattttgtagatgtagataggataatagaatccatagaaaatataggttatacagttatataaaaattactttaaaaattaataaaaacatggtaaaatataaatcgtataaagttgtgtaatttttaaggaggtgtgttacatatgcagtggcaagaactgtatcgccagagagtgtgctcagcggatgaagccgtagtggatagcctgaaaccgggaacgaaagtggtgtttggtcatgcagctgcggcccctgtaagattttctcaggctatgtatcgccaaagagaaaaactggaaaatattacagtgtttcatatgctttattttggcgatgcccctcaccttgcaccggaaatgcgtagccatgttcaccctactcttaattttcttgagggtaattctcgtccggccagccgcgatagacgtgtggattttataccgtgtcattttcacgaagttccagaactgtttcgccagggtttctttccattagatgttgcagttgtacaagtatctacccctaatgaagaaggatattgcagttttggcgtttcatgcgattatacgaaagcagctgcggaatgcgccccggtggttgtagcagaagtaaataaacagatgccatttattggtggagaaaatctgattcatatttcaaaacttacccacataattgaagtagatgaaccgatagctgaagtgcttccgccagcgattagtgatctggaattacgtattggtcagaattgtgcatcactgattaaagatggcgatacacttcaactgggtattggcggtataccagatgctgtgctgcgcgcgttagaaggtcataaagatttaggaattcacaccgaaatgtttacggatggagtaatgcgcatgataagaaaaggcataattaatggtaaaaagaaaacacttcatccagaaaaagtggttacgtctcttatttttggcagcaaagaactgtatgattttgtgaataataatccagttattgaatgttatcctgtggattatataaataatcctgatgttataggaaaaaatgatcgtatggtatctattaattcatgccttgaaatggatctgatgggccaggccgcaagtgaatcaataggatatgaacagttttctggcagcggaggccaagttgattttctgagaggtgctaaacgtagtaaaggtggaatatcaattatggcttttccttctacagcaaagaaaggtactgaaagccgcattgttccgatattaaaagaaggtgcttgcgttacaactggaagaaatgaagtagattatgtagtgactgaatatggcgtagcccgtttacgcggtgcaacacttagacaacgtgctgaagcgttaactgctatagcacatccagattttagaccagcattagaagaggaaatccgccgacgcttcgaataaggcatgcacggtagccaggaggttaatgaaagttacaaatcaaaaagaactaaaacaaaagctaaatgaattgagagaagcgcaaaagaagtttgcaacctatactcaagagcaagttgataaaatttttaaacaatgtgccatagccgcagctaaagaaagaataaacttagctaaattagcagtagaagaaacaggaataggtcttgtagaagataaaattataaaaaatcattttgcagcagaatatatatacaataaatataaaaatgaaaaaacttgtggcataatagaccatgacgattctttaggcataacaaaggttgctgaaccaattggaattgttgcagccatagttcctactactaatccaacttccacagcaattttcaaatcattaatttctttaaaaacaagaaacgcaatattcttttcaccacatccacgtgcaaaaaaatctacaattgctgcagcaaaattaattttagatgcagctgttaaagcaggagcacctaaaaatataataggctggatagatgagccatcaatagaactttctcaagatttgatgagtgaagctgatataatattagcaacaggaggtccttcaatggttaaagcggcctattcatctggaaaacctgcaattggtgttggagcaggaaatacaccagcaataatagatgagagtgcagatatagatatggcagtaagctccataattttatcaaagacttatgacaatggagtaatatgcgcttctgaacaatcaatattagttatgaattcaatatacgaaaaagttaaagaggaatttgtaaaacgaggatcatatatactcaatcaaaatgaaatagctaaaataaaagaaactatgtttaaaaatggagctattaatgctgacatagttggaaaatctgcttatataattgctaaaatggcaggaattgaagttcctcaaactacaaagatacttataggcgaagtacaatctgttgaaaaaagcgagctgttctcacatgaaaaactatcaccagtacttgcaatgtataaagttaaggattttgatgaagctctaaaaaaggcacaaaggctaatagaattaggtggaagtggacacacgtcatctttatatatagattcacaaaacaataaggataaagttaaagaatttggattagcaatgaaaacttcaaggacatttattaacatgccttcttcacagggagcaagcggagatttatacaattttgcgatagcaccatcatttactcttggatgcggcacttggggaggaaactctgtatcgcaaaatgtagagcctaaacatttattaaatattaaaagtgttgctgaaagaagggaaaatatgctttggtttaaagtgccacaaaaaatatattttaaatatggatgtcttagatttgcattaaaagaattaaaagatatgaataagaaaagagcctttatagtaacagataaagatctttttaaacttggatatgttaataaaataacaaaggtactagatgagatagatattaaatacagtatatttacagatattaaatctgatccaactattgattcagtaaaaaaaggtgctaaagaaatgcttaactttgaacctgatactataatctctattggtggtggatcgccaatggatgcagcaaaggttatgcacttgttatatgaatatccagaagcagaaattgaaaatctagctataaactttatggatataagaaagagaatatgcaatttccctaaattaggtacaaaggcgatttcagtagctattcctacaactgctggtaccggttcagaggcaacaccttttgcagttataactaatgatgaaacaggaatgaaataccctttaacttcttatgaattgaccccaaacatggcaataatagatactgaattaatgttaaatatgcctagaaaattaacagcagcaactggaatagatgcattagttcatgctatagaagcatatgtttcggttatggctacggattatactgatgaattagccttaagagcaataaaaatgatatttaaatatttgcctagagcctataaaaatgggactaacgacattgaagcaagagaaaaaatggcacatgcctctaatattgcggggatggcatttgcaaatgctttcttaggtgtatgccattcaatggctcataaacttggggcaatgcatcacgttccacatggaattgcttgtgctgtattaatagaagaagttattaaatataacgctacagactgtccaacaaagcaaacagcattccctcaatataaatctcctaatgctaagagaaaatatgctgaaattgcagagtatttgaatttaaagggtactagcgataccgaaaaggtaacagccttaatagaagctatttcaaagttaaagatagatttgagtattccacaaaatataagtgccgctggaataaataaaaaagatttttataatacgctagataaaatgtcagagcttgcttttgatgaccaatgtacaacagctaatcctaggtatccacttataagtgaacttaaggatatctatataaaatcattttaacccgggccccgggaacccagctttcttgtacaaagtggtgatctcgagaccttaagtaataaaaataagagttaccttaaatggtaactcttatttttttaatgtcgactcatagaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 2 - DNA, 15674 bp, Artificial
  Clostridium/E. coli shuttle vector pGn15_AKG(op)
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgcgccattcgccattcaggctgcgcaactgttgggaagggcgatcggtgcgggcctcttcgctattacgccagtctagacagggccccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgatcgggccccctaggcctctggcgaaagggggatgtgctgcaaggcgattaagttgggtaacgccagggttttcccagtcacgacgttgtaaaacgacggccagtgccaagcttgagcttaatcgcactggccggccgcttataatccataacaatcatcctttctgtgacactgtcagacacttatcacattaagtatatactattattaaactattctatatacttaatttattttaatagaaaaacataatatcataataacttcaaaattaaactttatttatgatttcatacttgactttgattttagaaaggatatactttttagcagatttggaaacggctttggacgtagtttgcccatagatgaacaaacaaactacatccaaaaattatacttttcccttcattggtatccgtatttttacatcttaatagcgtatgtattacaacacacctaaacaacgaccttacggtctgctactgcatatcctagcttgattgtttagttgcctcaactatgcttaaccctaccccgaactctttttttattgtgggttttcgtcgtgaagtcccaccgacacataatcataacataagatgtattatgaaaatgcgagtgactatccttttgtatcggctcactacaccacagatatattttttagtgcatactgtgtcggcactctcaatattaaattaataaataaattattttttctttttactcttctttacatgagcttttttaaagctccttgcataatatttaatgcatgtacgttcttttttctgttcttcctctgtaaaacatctcatttttatggcacaccatccatatcggttcatctttgaacaattaatacattggactttccctttatgtaaacatcttgactcattgtatttactgcaataggttgctactgttttatcatgaatcatcaacgaatgtattttgcatatttcagtatctttaataaactctttgcaatttttacaagttttcatatacgcccttcttttcataaattaatttatgaattctatgtattccaaggagctttttaaagctccccctttcgtactacttagctacaagcactataaaagtcataatgtttactgctaaagtcaataatgatattgctaataccttgttatttgataagatactgctttcctctgtcactttgctcaccccctttcattttcataaattaattatgaaaaaataaatatacttctaatttttatcaaataaaaaagcctttgcgtactgcttccaatacacaaaggctataaacttctaaatcttacttattgcaatttacatttatatctgttaagataatctcataaattgaatatatatagttaatgtttctcttgtatgtcggtacatttgaaatattgctatagatagagttctctaacggcttgatgtgttggtagcacatttaagttttggcttatatactaagttggtagcttaatatataagagctgaggacttatttttttattaaatttttcaacttgtctatattttaacccgtaattgaatacataacaagtatttttttttgtattcaattaaacattcataaatgagtataattaatcatactaaattctataattttcttttctgtaaatttctttctattcagcactgttatgccttttgactatcacttaataaaaaataagaaatgaattgtcaattgttcaaaaaaataatggcggcgcgccatgcaaaagacataatcgattcacaaaaaataggtacacgaaaaacaagttaagggatgcagtttatgcatcccttaacttacttattaaataatttatagctattgaaaagagataagaattgttcaaagctaatattgtttaaatcgtcaattcctgcatgttttaaggaattgttaaattgattttttgtaaatattttcttgtattctttgttaacccatttcataacgaaataattatacttctgtttatctttgtgtgatattcttgatttttttctatttaatctgataagtgagctattcactttaggtttaggatgaaaatattctcttggaaccatacttaatatagaaatatcaacttctgccattaaaaataatgccaatgagcgttttgtatttaataatcttttagcaaacccgtattccacgattaaataaatctcatcagctatactatcaaaaacaattttgcgtattatatccgtacttatgttataaggtatattaccaaatattttataggattggtttttaggaaatttaaactgcaatatatccttgtttaaaacttggaaattatcgtgatcaacaagtttattttctgtagttttgcataatttatggtctatttcaatggcagttacgaaattacacctctgtactaattcaagggtaaaatgcccttttcctgagccgatttcaaagatattatcatgttcatttaatcttatatttgtcattattttatctatattatgttttgaagtaataaagttttgactgtgttttatatttttctcgttcattataaccctctttattttttcctccttataaaattagtataattatagcacgagctctgataaatatgaacatgatgagtgatcgttaaatttatactgcaatctgatgcgattattgaataaaagatatgagagatttatctagtttctttttttacaagaaaaaagaaagttcttaaaggttttatacttttggtcgtagagcacacggtttaacgacttaattacgaagtaaataagtctagtgtgttagactttaatgtttttttaaggcattagtgcatttaagcgtcagagcatggctttatgccgagaaaactattggttggaatggcgtgtgtgttagccaaagctcctgcagtaactagtatcacaagtttgtacaaaaaagcaggcttaaggctcctaagtgaaatatatacatattataacaataaaataagtattagtgtaggatttttaaatagagtatctattttcagattaaatttttgcttatttgatttacattatataatattgagtaaagtattgactagcaaaattttttgatactttaatttgtgaaatttcttatcaaaagttatatttttgaatgatttttattgaaaaatacaactaaaaaggattatagtataagtgtgtgtaattttgtgttaaatttaagccagctgtaggagggaagcatgtacagaaaatttagagatgatcctagtagtgtagatcctagttggcatgaatttttagttgattatagtcctgaacctacatcacaacctgcagcagaacctacaagagttacatcacctttagttgcagaaagagcagcagcagcagcacctcaagcacctcctaaacctgcagatacagcagcagcaggaaatggagttgttgcagcattagcagcaaaaacagctgttcctcctcctgcagaaggagatgaagttgcagttttaagaggagcagcagctgcagttgttaaaaacatgagtgctagtttagaagttcctacagcaacaagtgttagagcagttcctgcaaaattactgatagacaacagaatagttataaacaatcaattaaaaagaacaagaggaggaaaaataagttttacacatttattaggatatgcattagttcaagcagttaaaaaatttcctaatatgaacagacactatacagaagtcgatggaaaacctacagcagttacacctgcacacacaaacttaggattagcgatagatctacaaggaaaagatggaaaaagaagtttagtagttgcaggtataaagagatgtgaaacaatgagatttgcacaatttgttacagcatatgaagatatagtacgtagagcaagagatggaaaattaacgacagaagattttgcaggagttacaataagtcttacaaatcctggaacaataggaacagttcatagtgttcctagattaatgccaggacaaggagcaataataggagttggagcaatggaatatcctgctgaatttcaaggagcatctgaagaaagaatagctgaattaggtattgggaaattaataaccttaacatctacatatgatcatagaataatacaaggagcagaaagtggagattttttaagaacgatacatgaattactgctcagtgatggattttgggatgaagtgtttagagaattgtccataccatatttacctgttagatggtctacagataatcctgatagtatagttgataaaaatgcaagagttatgaatttaatagcagcatatagaaatagaggacatttaatggcagatacagatcccttacgattagataaagcaagatttagaagtcatcctgatttagaagttttaacacatggattaacactttgggatttagatagagttttcaaagttgatggatttgcaggagcacaatataagaaactcagagatgtcttaggattgttaagagatgcatattgtcgccatataggagttgaatatgcacatatcttagatcctgaacaaaaggaatggttagaacaaagagttgagactaaacatgtcaaacctacagttgcacaacaaaagtatatcttaagcaaattaaatgcagctgaagcatttgaaacatttttacagacaaaatatgttggacagaaaagatttagtttagaaggagcagaaagtgttatacctatgatggatgcagcaatagatcaatgtgcagaacatggattagatgaagtagttataggaatgcctcatagaggaagactaaatgttctagcaaatatagttgggaaaccgtatagtcaaatattcacagaattcgaaggaaatctgaatccaagtcaagcacatggaagtggagatgttaaatatcatttaggagcaacaggattatacttacaaatgtttggagataacgatatacaagtttctttaacagcaaatcctagtcatttagaagcagttgaccctgttttagaaggattagttagagcaaaacaagatttactcgatcatggaagtatcgatagtgatggccaaagagcattttcagttgttccgttaatgttacatggagatgcagcatttgcaggacaaggagttgttgcagaaacactgaatttagcaaatttacctggatatagagttggaggaacaatacacatcatagttaataatcagataggatttacaacagcacctgaatatagtcgtagtagtgaatattgtacagatgttgcaaaaatgataggagctcctatatttcatgttaatggagatgatcctgaagcatgtgtttgggttgcaagattagcagttgattttagacaaagatttaagaaagatgttgttatagacatgctatgctatagaagaagaggacataacgaaggagatgatcctagtatgacaaatccttacatgtatgacgttgttgatacgaaaagaggagctagaaagagttatacagaagcattaataggaagaggagatataagtatgaaagaagcagaagatgcattaagggattatcaaggacaattagaaagagttttcaatgaagttagagaattagaaaaacatggcgttcaacctagtgaaagtgtagaaagtgaccaaatgattcctgcaggattagctacagcagttgataaaagtctgttagcaagaataggagatgcatttttagcattacctaatggatttactgcacatcctagagttcaacctgtgttagaaaagagaagagaaatggcatatgagggaaaaatagattgggcatttggagaactattagcattagggagtttagttgcagaaggaaaattagtgcgcttaagtggacaagatagtagaagaggtaccttcagtcaaagacacagtgttttaatagacagacatacaggtgaagagtttacaccgttacaattgttagcaacaaatagtgatggaagtcctacaggagggaaatttctcgtttatgatagtcctttaagtgaatatgcagcagttggatttgagtatggatatacagttggaaatcctgatgcagttgttttatgggaagcacaatttggagattttgttaatggagcacagagtataatcgacgaatttatatctagtggagaggcaaaatggggacaattaagcaatgtcgttttattgttacctcatggacatgaaggacaaggacctgatcatacaagtgcaagaatcgaaagatttttacaactatgggcagaaggaagtatgacaattgcaatgcctagtacaccttccaattactttcatcttttacgtagacatgcattagatggaattcaaagacccttaatagtttttacacccaaaagtatgcttagacataaagctgcagttagtgagataaaggactttacagaaatcaaatttcgaagtgttttagaagagcctacttatgaagatggaataggagatagaaataaagtttcaagaatattactcacaagtggaaagttatattatgagttagcagcaagaaaggcaaaagacaatagaaatgatttagcaatagttagattagaacaattagcacctttaccaagaagaagactaagagaaacattagatagatatgagaacgttaaagaattcttttgggttcaagaagaacctgcaaatcaaggagcatggcctagatttgggttagaattgcctgaattattacctgataaactagctggaataaaaagaatatcaagaagagcaatgagtgcacctagtagtggaagtagtaaagttcatgcagttgaacaacaagagatattagatgaagcatttggatagaagaggaggttaggcatgcagctgtttaaactcaagagtgtaacacatcatttcgatacttttgcagaatttgctaaagagttctgtttaggagaaagagatcttgtgatcactaacgaatttatctacgaaccatatatgaaagcttgtcaattaccttgtcactttgttatgcaggaaaagtatggtcaaggtgaacctagtgatgaaatgatgaacaatatactcgcagacattcgtaatattcagtttgatagagtcataggaataggaggaggtactgtgatagacatcagtaagctatttgttcttaaaggtctcaatgacgttttagatgcatttgatagaaaaatccccttaatcaaagagaaggaactcatcatagttcctacaacatgtggtacaggaagtgaagtaacgaatataagcatagctgaaatcaaatcaagacacactaaaatgggtttagctgatgatgcaatagtagcagatcatgcgattataatacccgaactacttaaatctctcccttttcatttttatgcttgtagtgctattgatgcattgattcatgcaattgagtcttatgtaagccctaaagcaagtccatatagcagattattcagtgaagcagcatgggatataatactcgaggtttttaaaaagattgctgaacatggaccagaatataggtttgagaagttaggagagatgataatggcttcgaattatgcaggtattgcatttggaaatgctggtgttggtgctgtacatgcattatcttaccctttaggtggaaattatcatgttccacatggagaagcaaactatcaattttttaccgaagtttttaaagtttatcaaaagaaaaatccgtttggatatatcgttgagttaaactggaaactttccaaaattctcaattgtcaacctgaatacgtatatcctaagctagatgaacttctcggttgtcttttgaccaaaaaacccttacatgaatatggaatgaaagatgaagaagttagaggatttgctgaatctgtgttaaagacgcaacaaagattattggctaacaattatgtcgaacttacagtagatgagatagaagggatttataggagactttactagagcttccgcggaggccggcgcggccgatttaaatcctcagcctgagcccccgggcggtatcagctcactcaaaggcggtaataacaactttgtatacaaaagttgtagtgtagtagcctgtgaaataagtaaggaaaaaaaagaagtaagtgttatatatgatgattattttgtagatgtagataggataatagaatccatagaaaatataggttatacagttatataaaaattactttaaaaattaataaaaacatggtaaaatataaatcgtataaagttgtgtaatttttaaggaggtgtgttacatatgcagtggcaagaactgtatcgccagagagtgtgctcagcggatgaagccgtagtggatagcctgaaaccgggaacgaaagtggtgtttggtcatgcagctgcggcccctgtaagattttctcaggctatgtatcgccaaagagaaaaactggaaaatattacagtgtttcatatgctttattttggcgatgcccctcaccttgcaccggaaatgcgtagccatgttcaccctactcttaattttcttgagggtaattctcgtccggccagccgcgatagacgtgtggattttataccgtgtcattttcacgaagttccagaactgtttcgccagggtttctttccattagatgttgcagttgtacaagtatctacccctaatgaagaaggatattgcagttttggcgtttcatgcgattatacgaaagcagctgcggaatgcgccccggtggttgtagcagaagtaaataaacagatgccatttattggtggagaaaatctgattcatatttcaaaacttacccacataattgaagtagatgaaccgatagctgaagtgcttccgccagcgattagtgatctggaattacgtattggtcagaattgtgcatcactgattaaagatggcgatacacttcaactgggtattggcggtataccagatgctgtgctgcgcgcgttagaaggtcataaagatttaggaattcacaccgaaatgtttacggatggagtaatgcgcatgataagaaaaggcataattaatggtaaaaagaaaacacttcatccagaaaaagtggttacgtctcttatttttggcagcaaagaactgtatgattttgtgaataataatccagttattgaatgttatcctgtggattatataaataatcctgatgttataggaaaaaatgatcgtatggtatctattaattcatgccttgaaatggatctgatgggccaggccgcaagtgaatcaataggatatgaacagttttctggcagcggaggccaagttgattttctgagaggtgctaaacgtagtaaaggtggaatatcaattatggcttttccttctacagcaaagaaaggtactgaaagccgcattgttccgatattaaaagaaggtgcttgcgttacaactggaagaaatgaagtagattatgtagtgactgaatatggcgtagcccgtttacgcggtgcaacacttagacaacgtgctgaagcgttaactgctatagcacatccagattttagaccagcattagaagaggaaatccgccgacgcttcgaataaggcatgcacggtagccaggaggttaatgaaagttacaaatcaaaaagaactaaaacaaaagctaaatgaattgagagaagcgcaaaagaagtttgcaacctatactcaagagcaagttgataaaatttttaaacaatgtgccatagccgcagctaaagaaagaataaacttagctaaattagcagtagaagaaacaggaataggtcttgtagaagataaaattataaaaaatcattttgcagcagaatatatatacaataaatataaaaatgaaaaaacttgtggcataatagaccatgacgattctttaggcataacaaaggttgctgaaccaattggaattgttgcagccatagttcctactactaatccaacttccacagcaattttcaaatcattaatttctttaaaaacaagaaacgcaatattcttttcaccacatccacgtgcaaaaaaatctacaattgctgcagcaaaattaattttagatgcagctgttaaagcaggagcacctaaaaatataataggctggatagatgagccatcaatagaactttctcaagatttgatgagtgaagctgatataatattagcaacaggaggtccttcaatggttaaagcggcctattcatctggaaaacctgcaattggtgttggagcaggaaatacaccagcaataatagatgagagtgcagatatagatatggcagtaagctccataattttatcaaagacttatgacaatggagtaatatgcgcttctgaacaatcaatattagttatgaattcaatatacgaaaaagttaaagaggaatttgtaaaacgaggatcatatatactcaatcaaaatgaaatagctaaaataaaagaaactatgtttaaaaatggagctattaatgctgacatagttggaaaatctgcttatataattgctaaaatggcaggaattgaagttcctcaaactacaaagatacttataggcgaagtacaatctgttgaaaaaagcgagctgttctcacatgaaaaactatcaccagtacttgcaatgtataaagttaaggattttgatgaagctctaaaaaaggcacaaaggctaatagaattaggtggaagtggacacacgtcatctttatatatagattcacaaaacaataaggataaagttaaagaatttggattagcaatgaaaacttcaaggacatttattaacatgccttcttcacagggagcaagcggagatttatacaattttgcgatagcaccatcatttactcttggatgcggcacttggggaggaaactctgtatcgcaaaatgtagagcctaaacatttattaaatattaaaagtgttgctgaaagaagggaaaatatgctttggtttaaagtgccacaaaaaatatattttaaatatggatgtcttagatttgcattaaaagaattaaaagatatgaataagaaaagagcctttatagtaacagataaagatctttttaaacttggatatgttaataaaataacaaaggtactagatgagatagatattaaatacagtatatttacagatattaaatctgatccaactattgattcagtaaaaaaaggtgctaaagaaatgcttaactttgaacctgatactataatctctattggtggtggatcgccaatggatgcagcaaaggttatgcacttgttatatgaatatccagaagcagaaattgaaaatctagctataaactttatggatataagaaagagaatatgcaatttccctaaattaggtacaaaggcgatttcagtagctattcctacaactgctggtaccggttcagaggcaacaccttttgcagttataactaatgatgaaacaggaatgaaataccctttaacttcttatgaattgaccccaaacatggcaataatagatactgaattaatgttaaatatgcctagaaaattaacagcagcaactggaatagatgcattagttcatgctatagaagcatatgtttcggttatggctacggattatactgatgaattagccttaagagcaataaaaatgatatttaaatatttgcctagagcctataaaaatgggactaacgacattgaagcaagagaaaaaatggcacatgcctctaatattgcggggatggcatttgcaaatgctttcttaggtgtatgccattcaatggctcataaacttggggcaatgcatcacgttccacatggaattgcttgtgctgtattaatagaagaagttattaaatataacgctacagactgtccaacaaagcaaacagcattccctcaatataaatctcctaatgctaagagaaaatatgctgaaattgcagagtatttgaatttaaagggtactagcgataccgaaaaggtaacagccttaatagaagctatttcaaagttaaagatagatttgagtattccacaaaatataagtgccgctggaataaataaaaaagatttttataatacgctagataaaatgtcagagcttgcttttgatgaccaatgtacaacagctaatcctaggtatccacttataagtgaacttaaggatatctatataaaatcattttaacccgggccccgggaacccagctttcttgtacaaagtggtgatctcgagaccttaagtaataaaaataagagttaccttaaatggtaactcttatttttttaatgtcgactcatagaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 3 - DNA, 15547 bp, Artificial
  Clostridium/E. coli shuttle vector pM9_SUC(op)
acaggttggctgataagtccccggtctgaagttcctattctctagaaagtataggaacttcttccgacgcttattcgcttcgctcatatttatatagaaaagaagtgaatgcgcaaaagacataatcgattcacaaaaaataggtacacgaaaaacaagttaagggatgcagtttatgcatcccttaacttacttattaaataatttatagctattgaaaagagataagaattgttcaaagctaatattgtttaaatcgtcaattcctgcatgttttaaggaattgttaaattgattttttgtaaatattttcttgtattctttgttaacccatttcataacgaaataattatacttctgtttatctttgtgtgatattcttgatttttttctatttaatctgataagtgagctattcactttaggtttaggatgaaaatattctcttggaaccatacttaatatagaaatatcaacttctgccattaaaaataatgccaatgagcgttttgtatttaataatcttttagcaaacccgtattccacgattaaataaatctcatcagctatactatcaaaaacaattttgcgtattatatccgtacttatgttataaggtatattaccaaatattttataggattggtttttaggaaatttaaactgcaatatatccttgtttaaaacttggaaattatcgtgatcaacaagtttattttctgtagttttgcataatttatggtctatttcaatggcagttacgaaattacacctctgtactaattcaagggtaaaatgcccttttcctgagccgatttcaaagatattatcatgttcatttaatcttatatttgtcattattttatctatattatgttttgaagtaataaagttttgactgtgttttatatttttctcgttcattataaccctctttattttttcctccttataaaattagtataattatagcacgagctctgataaatatgaacatgatgagtgatcgttaaatttatactgcaatctgatgcgattattgaagttcctattctctagaaagtataggaacttcacggcagtgacggtttccctctagatcacaagtttgtacaaaaaagcaggcttaaggctcctaagtgaaatatatacatattataacaataaaataagtattagtgtaggatttttaaatagagtatctattttcagattaaatttttgcttatttgatttacattatataatattgagtaaagtattgactagcaaaattttttgatactttaatttgtgaaatttcttatcaaaagttatatttttgaatgatttttattgaaaaatacaactaaaaaggattatagtataagtgtgtgtaattttgtgttaaatttaaggcagcgttccaggagggacagaacaatgaacttacatgaatatcaggcaaaacaactttttgcccgctatggcttaccagcaccggtgggttatgcctgtactactccgcgcgaagcagaagaagccgcttcaaaaatcggtgccggtccgtgggtagtgaaatgtcaggttcacgctggtggccgcggtaaagcgggcggtgtgaaagttgtaaacagcaaagaagacatccgtgcttttgcagaaaactggctgggcaagcgtctggtaacgtatcaaacagatgccaatggccaaccggttaaccagattctggttgaagcagcgaccgatatcgctaaagagctgtatctcggtgccgttgttgaccgtagttcccgtcgtgtggtctttatggcctccaccgaaggcggcgtggaaatcgaaaaagtggcggaagaaactccgcacctgatccataaagttgcgcttgatccgctgactggcccgatgccgtatcagggacgcgagctggcgttcaaactgggtctggaaggtaaactggttcagcagttcaccaaaatcttcatgggcctggcgaccattttcctggagcgcgacctggcgttgatcgaaatcaacccgctggtcatcaccaaacagggcgatctgatttgcctcgacggcaaactgggcgctgacggcaacgcactgttccgccagcctgatctgcgcgaaatgcgtgaccagtcgcaggaagatccgcgtgaagcacaggctgcacagtgggaactgaactacgttgcgctggacggtaacatcggttgtatggttaacggcgcaggtctggcgatgggtacgatggacatcgttaaactgcacggcggcgaaccggctaacttccttgacgttggcggcggcgcaaccaaagaacgtgtaaccgaagcgttcaaaatcatcctctctgacgacaaagtgaaagccgttctggttaacatcttcggcggtatcgttcgttgcgacctgatcgctgacggtatcatcggcgcggtagcagaagtgggtgttaacgtaccggtcgtggtacgtctggaaggtaacaacgccgaactcggcgcgaagaaactggctgacagcggcctgaatattattgcagcaaaaggtctgacggatgcagctcagcaggttgttgccgcagtggaggggaaataacaaggcaaagtgaggaggtaaggtatgagtaatgaagtatctataaaagaattaattgaaaaggcaaaggtggcacaaaaaaaattggaagcctatagtcaagaacaagttgatgtactagtaaaagcactaggaaaagtggtttatgataatgcagaaatgtttgcaaaagaagcagttgaagaaacagaaatgggtgtttatgaagataaagtagctaaatgtcatttgaaatcaggagctatttggaatcatataaaagacaagaaaactgtaggcataataaaagaagaacctgaaagggcacttgtttatgttgctaagccaaagggagttgtggcagctactacgcctataactaatccagtggtaactcctatgtgtaatgcaatggctgctataaagggcagaaatacaataatagtagcaccacatcctaaagcaaagaaagtttcagctcatactgtagaacttatgaatgctgagcttaaaaaattgggagcaccagaaaatatcatacagatagtagaagcaccatcaagagaagctgctaaggaacttatggaaagtgctgatgtagttattgctacaggcggtgctggaagagttaaagctgcttactccagtggaagaccagcttatggcgttggacctggaaattcacaggtaatagttgataagggatacgattataacaaagctgcacaggatataataacaggaagaaaatatgacaatggaattatatgttcttcagagcaatcagttatagctcctgctgaagattatgataaggtaatagcagcttttgtagaaaatggggcattctatgtagaagatgaggaaacagtagaaaagtttagatcaactttatttaaagatggaaaaataaacagcaagattataggtaaatccgtccaaattattgcggatcttgcaggagtaaaagtaccagaaggtactaaggttatagtacttaagggtaaaggtgcaggagaaaaagatgtactttgtaaagaaaaaatgtgtccagttttagtagcattgaaatatgatacttttgaagaagcagttgaaatagctatggctaattatatgtatgaaggagctggtcatacagcaggcatacattctgacaatgacgagaacataagatatgcaggaactgtattacctataagcagattagttgtaaatcagcctgcaactactgctggaggaagtttcaataatggatttaaccctactactacactaggctgcggatcatggggcagaaacagtatttcagaaaatcttacttacgagcatcttataaatgtttcaagaatagggtatttcaataaagaagcaaaagttcctagctatgaggaaatatggggataaaagaggaggttaggcatgcagctgtttaaactcaagagtgtaacacatcatttcgatacttttgcagaatttgctaaagagttctgtttaggagaaagagatcttgtgatcactaacgaatttatctacgaaccatatatgaaagcttgtcaattaccttgtcactttgttatgcaggaaaagtatggtcaaggtgaacctagtgatgaaatgatgaacaatatactcgcagacattcgtaatattcagtttgatagagtcataggaataggaggaggtactgtgatagacatcagtaagctatttgttcttaaaggtctcaatgacgttttagatgcatttgatagaaaaatccccttaatcaaagagaaggaactcatcatagttcctacaacatgtggtacaggaagtgaagtaacgaatataagcatagctgaaatcaaatcaagacacactaaaatgggtttagctgatgatgcaatagtagcagatcatgcgattataatacccgaactacttaaatctctcccttttcatttttatgcttgtagtgctattgatgcattgattcatgcaattgagtcttatgtaagccctaaagcaagtccatatagcagattattcagtgaagcagcatgggatataatactcgaggtttttaaaaagattgctgaacatggaccagaatataggtttgagaagttaggagagatgataatggcttcgaattatgcaggtattgcatttggaaatgctggtgttggtgctgtacatgcattatcttaccctttaggtggaaattatcatgttccacatggagaagcaaactatcaattttttaccgaagtttttaaagtttatcaaaagaaaaatccgtttggatatatcgttgagttaaactggaaactttccaaaattctcaattgtcaacctgaatacgtatatcctaagctagatgaacttctcggttgtcttttgaccaaaaaacccttacatgaatatggaatgaaagatgaagaagttagaggatttgctgaatctgtgttaaagacgcaacaaagattattggctaacaattatgtcgaacttacagtagatgagatagaagggatttataggagactttactagagcttccgcggaggccggcgcggccgatttaaatcctcagcctgagcccccgggcggtatcagctcactcaaaggcggtaataacaactttgtatacaaaagttgtagtgtagtagcctgtgaaataagtaaggaaaaaaaagaagtaagtgttatatatgatgattattttgtagatgtagataggataatagaatccatagaaaatataggttatacagttatataaaaattactttaaaaattaataaaaacatggtaaaatataaatcgtataaagttgtgtaatttttaaggaggtgtgttacatatgcagtggcaagaactgtatcgccagagagtgtgctcagcggatgaagccgtagtggatagcctgaaaccgggaacgaaagtggtgtttggtcatgcagctgcggcccctgtaagattttctcaggctatgtatcgccaaagagaaaaactggaaaatattacagtgtttcatatgctttattttggcgatgcccctcaccttgcaccggaaatgcgtagccatgttcaccctactcttaattttcttgagggtaattctcgtccggccagccgcgatagacgtgtggattttataccgtgtcattttcacgaagttccagaactgtttcgccagggtttctttccattagatgttgcagttgtacaagtatctacccctaatgaagaaggatattgcagttttggcgtttcatgcgattatacgaaagcagctgcggaatgcgccccggtggttgtagcagaagtaaataaacagatgccatttattggtggagaaaatctgattcatatttcaaaacttacccacataattgaagtagatgaaccgatagctgaagtgcttccgccagcgattagtgatctggaattacgtattggtcagaattgtgcatcactgattaaagatggcgatacacttcaactgggtattggcggtataccagatgctgtgctgcgcgcgttagaaggtcataaagatttaggaattcacaccgaaatgtttacggatggagtaatgcgcatgataagaaaaggcataattaatggtaaaaagaaaacacttcatccagaaaaagtggttacgtctcttatttttggcagcaaagaactgtatgattttgtgaataataatccagttattgaatgttatcctgtggattatataaataatcctgatgttataggaaaaaatgatcgtatggtatctattaattcatgccttgaaatggatctgatgggccaggccgcaagtgaatcaataggatatgaacagttttctggcagcggaggccaagttgattttctgagaggtgctaaacgtagtaaaggtggaatatcaattatggcttttccttctacagcaaagaaaggtactgaaagccgcattgttccgatattaaaagaaggtgcttgcgttacaactggaagaaatgaagtagattatgtagtgactgaatatggcgtagcccgtttacgcggtgcaacacttagacaacgtgctgaagcgttaactgctatagcacatccagattttagaccagcattagaagaggaaatccgccgacgcttcgaataaggcatgcacggtagccaggaggttaatgaaagttacaaatcaaaaagaactaaaacaaaagctaaatgaattgagagaagcgcaaaagaagtttgcaacctatactcaagagcaagttgataaaatttttaaacaatgtgccatagccgcagctaaagaaagaataaacttagctaaattagcagtagaagaaacaggaataggtcttgtagaagataaaattataaaaaatcattttgcagcagaatatatatacaataaatataaaaatgaaaaaacttgtggcataatagaccatgacgattctttaggcataacaaaggttgctgaaccaattggaattgttgcagccatagttcctactactaatccaacttccacagcaattttcaaatcattaatttctttaaaaacaagaaacgcaatattcttttcaccacatccacgtgcaaaaaaatctacaattgctgcagcaaaattaattttagatgcagctgttaaagcaggagcacctaaaaatataataggctggatagatgagccatcaatagaactttctcaagatttgatgagtgaagctgatataatattagcaacaggaggtccttcaatggttaaagcggcctattcatctggaaaacctgcaattggtgttggagcaggaaatacaccagcaataatagatgagagtgcagatatagatatggcagtaagctccataattttatcaaagacttatgacaatggagtaatatgcgcttctgaacaatcaatattagttatgaattcaatatacgaaaaagttaaagaggaatttgtaaaacgaggatcatatatactcaatcaaaatgaaatagctaaaataaaagaaactatgtttaaaaatggagctattaatgctgacatagttggaaaatctgcttatataattgctaaaatggcaggaattgaagttcctcaaactacaaagatacttataggcgaagtacaatctgttgaaaaaagcgagctgttctcacatgaaaaactatcaccagtacttgcaatgtataaagttaaggattttgatgaagctctaaaaaaggcacaaaggctaatagaattaggtggaagtggacacacgtcatctttatatatagattcacaaaacaataaggataaagttaaagaatttggattagcaatgaaaacttcaaggacatttattaacatgccttcttcacagggagcaagcggagatttatacaattttgcgatagcaccatcatttactcttggatgcggcacttggggaggaaactctgtatcgcaaaatgtagagcctaaacatttattaaatattaaaagtgttgctgaaagaagggaaaatatgctttggtttaaagtgccacaaaaaatatattttaaatatggatgtcttagatttgcattaaaagaattaaaagatatgaataagaaaagagcctttatagtaacagataaagatctttttaaacttggatatgttaataaaataacaaaggtactagatgagatagatattaaatacagtatatttacagatattaaatctgatccaactattgattcagtaaaaaaaggtgctaaagaaatgcttaactttgaacctgatactataatctctattggtggtggatcgccaatggatgcagcaaaggttatgcacttgttatatgaatatccagaagcagaaattgaaaatctagctataaactttatggatataagaaagagaatatgcaatttccctaaattaggtacaaaggcgatttcagtagctattcctacaactgctggtaccggttcagaggcaacaccttttgcagttataactaatgatgaaacaggaatgaaataccctttaacttcttatgaattgaccccaaacatggcaataatagatactgaattaatgttaaatatgcctagaaaattaacagcagcaactggaatagatgcattagttcatgctatagaagcatatgtttcggttatggctacggattatactgatgaattagccttaagagcaataaaaatgatatttaaatatttgcctagagcctataaaaatgggactaacgacattgaagcaagagaaaaaatggcacatgcctctaatattgcggggatggcatttgcaaatgctttcttaggtgtatgccattcaatggctcataaacttggggcaatgcatcacgttccacatggaattgcttgtgctgtattaatagaagaagttattaaatataacgctacagactgtccaacaaagcaaacagcattccctcaatataaatctcctaatgctaagagaaaatatgctgaaattgcagagtatttgaatttaaagggtactagcgataccgaaaaggtaacagccttaatagaagctatttcaaagttaaagatagatttgagtattccacaaaatataagtgccgctggaataaataaaaaagatttttataatacgctagataaaatgtcagagcttgcttttgatgaccaatgtacaacagctaatcctaggtatccacttataagtgaacttaaggatatctatataaaatcattttaacccgggccccgggaacccagctttcttgtacaaagtggtgatgatccggctgctaacctcgagaacatgtgagcaataaaaataagaagcctgcatttgcaggcttcttatttttatagaccggggacttatcagccaacctgtaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacggtcctgcaggtaatacgactcactatagggataacttaaatttatattttttactttataatatataattgattatagaatccaaattagataggaggaccaggtaaatggagaataattttattgtcaacgaaaacgagaaaagggttttaaagcagatatttaacaactccaatatcagtagaactcaaatttccaagaatttagaactgaataaagcaacaatttcgaacattcttaacaatctaaagcataaaagtttggtaaatgaagttggtgaaggaaattctactaaaagtggtggaagaaaacctatacttctcgaaattaatcaaaagtatgggtattatataagcatggatttgacatatgattctgttgagttaatgtataactattttgatgctacaattctcaaacaagatagttatgaactgaatgataagaatgtaagtagcatattacagatactcaaatctaacataaatgttagcgaaaaatacgacacattatacggtttattagggataagtatatccatacatgggattgtagatgatgaacaaaacatcattaatctaccatttcacaagaatgaaaaaagaacgtttactgatgaacttaagtctttcactaatgtaccagtggttattgaaaatgaggcaaatttaagtgcattatacgagaaaagtttatacatcaattcgaatattaacaacttgatcacattgtctatacataaaggtataggagctggcatcataatcaataagaaactctatagaggtagtaatggagaagctggagaaataggaaagactcttgttttagagtctatcaataacaatgataacaagtactataaaatagaggatatttgtagtcaagacgctttgatacaaaagattaataacagattaggagttacacttacatttaccgaactaatacagtattataacgaaggaaatagtattgtagctcatgaaatcaaacagtttatcaacaaaatgactgtcttaatacacaatcttaatactcagtttaatccagatgcaatatacatcaattgtcccttgattaatgaacttcctaatattctaaacgaaatcaaagaacaattctcttgtttttcacaaggaagtcctatacaattgcatttaaccactaatgttaaacaagctacattattaggtggaacattagcaattatgcagaaaacccttaacataaacaatatccaaatgaatattaaatagttacagcagtctgagttataaaatagatatctcggaccgtctattcttccttatttgagtgggtaccaatgaaatctttcgagctaaatcgatttctggcccactcttttttattttttagtaagcgtttacaaaaaatgaacaatgtgctatattacctctaaagttagtttgtttattaaattaaccaactaaaatgtcgggatccaattaggaggaatctgcgatggaaaagaaggaatttagagttttaataaagtattgttttcttaaaggaaagaatacagtagaagctaagacttggttagataatgaatttccagattctgctcctggtaaaagtacaattatagattggtatgcaaaatttaaaagaggagaaatgtcaacagaagatggagaaagatctggtagaccaaaagaagttgtaactgatgaaaatataaagaagatacataaaatgatattaaatgatagaaagatgaagttaatagaaatagcagaagctttaaaaatatctaaagaaagagttggacatataatacatcaatatttagatatgagaaaattatgtgcaaaatgggttccaagagaattaacttttgatcaaaagcaaagaagagtagatgatagtaagagatgtttacaattattaacaagaaatactccagaatttttcagaagatatgtaacaatggatgaaacttggttacatcattatacacctgaaagtaatagacaatcagcagaatggacagctactggagaaccatcacctaaaagaggaaagactcaaaagagtgctggaaaggttatggcatcagtattttgggatgctcatggtataatatttatagattatttagaaaaaggaaaaacaataaattctgattattatatggcattattagaaagattaaaagttgaaatagcagctaaaagaccacatatgaagaagaaaaaagtattatttcatcaagataatgctccttgtcataagtcattaagaactatggcaaagatacatgaattaggttttgaattattaccacatccaccttattcaccagatttagctccttctgatttctttttatttagtgatttaaagagaatgttagcaggaaagaagtttggttgtaatgaagaagttatagcagaaacagaagcttattttgaagcaaaacctaaggaatattatcaaaatggaataaagaagttagaaggtagatataatagatgtatagcattagaaggaaattatgtagaataagaattccttaattaaggagctcgataaaaataagaagcctgcatttgcaggcttcttatttttatggtcatagctgtttcctggcgcgccgcattcacttcttttctatataaatatgagcgaagcgaataagcgtcggaaaagcagcaaaaagtttcctttttgctgttggagcatgggggttcagggggtgcagtatctgacgtcaatgccgagcgaaagcgagccgaagggtagcatttacgttagataaccccctgatatgctccgacgctttatatagaaaagaagattcaactaggtaaaatcttaatataggttgagatgataaggtttataaggaatttgtttgttctaatttttcactcattttgttctaatttcttttaacaaatgttcttttttttttagaacagttatgatatagttagaatagtttaaaataaggagtgagaaaaagatgaaagaaagatatggaacagtctataaaggctctcagaggctcatagacgaagaaagtggagaagtcatagaggtagacaagttataccgtaaacaaacgtctggtaacttcgtaaaggcatatatagtgcaattaataagtatgttagatatgattggcggaaaaaaacttaaaatcgttaactatatcctagataatgtccacttaagtaacaatacaatgatagctacaacaagagaaatagcaaaagctacaggaacaagtctacaaacagtaataacaacacttaaaatcttagaagaaggaaatattataaaaagaaaaactggagtattaatgttaaaccctgaactactaatgagaggcgacgaccaaaaacaaaaatacctcttactcgaatttgggaactttgagcaagaggcaaatgaaatagattgacctcccaataacaccacgtagttattgggaggtcaatctatgaaatgcgattaagggccggccagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaacaagtatttaagcaaaaacatcgtagaaatacggtgttttttgttaccctaagttt

SEQ ID NO: 4 - DNA, 16625 bp, Artificial
  Clostridium/E. coli shuttle vector pM9_AKG
acaggttggctgataagtccccggtctgaagttcctattctctagaaagtataggaacttcttccgacgcttattcgcttcgctcatatttatatagaaaagaagtgaatgcgcaaaagacataatcgattcacaaaaaataggtacacgaaaaacaagttaagggatgcagtttatgcatcccttaacttacttattaaataatttatagctattgaaaagagataagaattgttcaaagctaatattgtttaaatcgtcaattcctgcatgttttaaggaattgttaaattgattttttgtaaatattttcttgtattctttgttaacccatttcataacgaaataattatacttctgtttatctttgtgtgatattcttgatttttttctatttaatctgataagtgagctattcactttaggtttaggatgaaaatattctcttggaaccatacttaatatagaaatatcaacttctgccattaaaaataatgccaatgagcgttttgtatttaataatcttttagcaaacccgtattccacgattaaataaatctcatcagctatactatcaaaaacaattttgcgtattatatccgtacttatgttataaggtatattaccaaatattttataggattggtttttaggaaatttaaactgcaatatatccttgtttaaaacttggaaattatcgtgatcaacaagtttattttctgtagttttgcataatttatggtctatttcaatggcagttacgaaattacacctctgtactaattcaagggtaaaatgcccttttcctgagccgatttcaaagatattatcatgttcatttaatcttatatttgtcattattttatctatattatgttttgaagtaataaagttttgactgtgttttatatttttctcgttcattataaccctctttattttttcctccttataaaattagtataattatagcacgagctctgataaatatgaacatgatgagtgatcgttaaatttatactgcaatctgatgcgattattgaagttcctattctctagaaagtataggaacttcacggcagtgacggtttccctctagatcacaagtttgtacaaaaaagcaggcttaaggctcctaagtgaaatatatacatattataacaataaaataagtattagtgtaggatttttaaatagagtatctattttcagattaaatttttgcttatttgatttacattatataatattgagtaaagtattgactagcaaaattttttgatactttaatttgtgaaatttcttatcaaaagttatatttttgaatgatttttattgaaaaatacaactaaaaaggattatagtataagtgtgtgtaattttgtgttaaatttaagccagctgtaggagggaagcatgtacagaaaatttagagatgatcctagtagtgtagatcctagttggcatgaatttttagttgattatagtcctgaacctacatcacaacctgcagcagaacctacaagagttacatcacctttagttgcagaaagagcagcagcagcagcacctcaagcacctcctaaacctgcagatacagcagcagcaggaaatggagttgttgcagcattagcagcaaaaacagctgttcctcctcctgcagaaggagatgaagttgcagttttaagaggagcagcagctgcagttgttaaaaacatgagtgctagtttagaagttcctacagcaacaagtgttagagcagttcctgcaaaattactgatagacaacagaatagttataaacaatcaattaaaaagaacaagaggaggaaaaataagttttacacatttattaggatatgcattagttcaagcagttaaaaaatttcctaatatgaacagacactatacagaagtcgatggaaaacctacagcagttacacctgcacacacaaacttaggattagcgatagatctacaaggaaaagatggaaaaagaagtttagtagttgcaggtataaagagatgtgaaacaatgagatttgcacaatttgttacagcatatgaagatatagtacgtagagcaagagatggaaaattaacgacagaagattttgcaggagttacaataagtcttacaaatcctggaacaataggaacagttcatagtgttcctagattaatgccaggacaaggagcaataataggagttggagcaatggaatatcctgctgaatttcaaggagcatctgaagaaagaatagctgaattaggtattgggaaattaataaccttaacatctacatatgatcatagaataatacaaggagcagaaagtggagattttttaagaacgatacatgaattactgctcagtgatggattttgggatgaagtgtttagagaattgtccataccatatttacctgttagatggtctacagataatcctgatagtatagttgataaaaatgcaagagttatgaatttaatagcagcatatagaaatagaggacatttaatggcagatacagatcccttacgattagataaagcaagatttagaagtcatcctgatttagaagttttaacacatggattaacactttgggatttagatagagttttcaaagttgatggatttgcaggagcacaatataagaaactcagagatgtcttaggattgttaagagatgcatattgtcgccatataggagttgaatatgcacatatcttagatcctgaacaaaaggaatggttagaacaaagagttgagactaaacatgtcaaacctacagttgcacaacaaaagtatatcttaagcaaattaaatgcagctgaagcatttgaaacatttttacagacaaaatatgttggacagaaaagatttagtttagaaggagcagaaagtgttatacctatgatggatgcagcaatagatcaatgtgcagaacatggattagatgaagtagttataggaatgcctcatagaggaagactaaatgttctagcaaatatagttgggaaaccgtatagtcaaatattcacagaattcgaaggaaatctgaatccaagtcaagcacatggaagtggagatgttaaatatcatttaggagcaacaggattatacttacaaatgtttggagataacgatatacaagtttctttaacagcaaatcctagtcatttagaagcagttgaccctgttttagaaggattagttagagcaaaacaagatttactcgatcatggaagtatcgatagtgatggccaaagagcattttcagttgttccgttaatgttacatggagatgcagcatttgcaggacaaggagttgttgcagaaacactgaatttagcaaatttacctggatatagagttggaggaacaatacacatcatagttaataatcagataggatttacaacagcacctgaatatagtcgtagtagtgaatattgtacagatgttgcaaaaatgataggagctcctatatttcatgttaatggagatgatcctgaagcatgtgtttgggttgcaagattagcagttgattttagacaaagatttaagaaagatgttgttatagacatgctatgctatagaagaagaggacataacgaaggagatgatcctagtatgacaaatccttacatgtatgacgttgttgatacgaaaagaggagctagaaagagttatacagaagcattaataggaagaggagatataagtatgaaagaagcagaagatgcattaagggattatcaaggacaattagaaagagttttcaatgaagttagagaattagaaaaacatggcgttcaacctagtgaaagtgtagaaagtgaccaaatgattcctgcaggattagctacagcagttgataaaagtctgttagcaagaataggagatgcatttttagcattacctaatggatttactgcacatcctagagttcaacctgtgttagaaaagagaagagaaatggcatatgagggaaaaatagattgggcatttggagaactattagcattagggagtttagttgcagaaggaaaattagtgcgcttaagtggacaagatagtagaagaggtaccttcagtcaaagacacagtgttttaatagacagacatacaggtgaagagtttacaccgttacaattgttagcaacaaatagtgatggaagtcctacaggagggaaatttctcgtttatgatagtcctttaagtgaatatgcagcagttggatttgagtatggatatacagttggaaatcctgatgcagttgttttatgggaagcacaatttggagattttgttaatggagcacagagtataatcgacgaatttatatctagtggagaggcaaaatggggacaattaagcaatgtcgttttattgttacctcatggacatgaaggacaaggacctgatcatacaagtgcaagaatcgaaagatttttacaactatgggcagaaggaagtatgacaattgcaatgcctagtacaccttccaattactttcatcttttacgtagacatgcattagatggaattcaaagacccttaatagtttttacacccaaaagtatgcttagacataaagctgcagttagtgagataaaggactttacagaaatcaaatttcgaagtgttttagaagagcctacttatgaagatggaataggagatagaaataaagtttcaagaatattactcacaagtggaaagttatattatgagttagcagcaagaaaggcaaaagacaatagaaatgatttagcaatagttagattagaacaattagcacctttaccaagaagaagactaagagaaacattagatagatatgagaacgttaaagaattcttttgggttcaagaagaacctgcaaatcaaggagcatggcctagatttgggttagaattgcctgaattattacctgataaactagctggaataaaaagaatatcaagaagagcaatgagtgcacctagtagtggaagtagtaaagttcatgcagttgaacaacaagagatattagatgaagcatttggatagaagaggaggttaggcatgcagctgtttaaactcaagagtgtaacacatcatttcgatacttttgcagaatttgctaaagagttctgtttaggagaaagagatcttgtgatcactaacgaatttatctacgaaccatatatgaaagcttgtcaattaccttgtcactttgttatgcaggaaaagtatggtcaaggtgaacctagtgatgaaatgatgaacaatatactcgcagacattcgtaatattcagtttgatagagtcataggaataggaggaggtactgtgatagacatcagtaagctatttgttcttaaaggtctcaatgacgttttagatgcatttgatagaaaaatccccttaatcaaagagaaggaactcatcatagttcctacaacatgtggtacaggaagtgaagtaacgaatataagcatagctgaaatcaaatcaagacacactaaaatgggtttagctgatgatgcaatagtagcagatcatgcgattataatacccgaactacttaaatctctcccttttcatttttatgcttgtagtgctattgatgcattgattcatgcaattgagtcttatgtaagccctaaagcaagtccatatagcagattattcagtgaagcagcatgggatataatactcgaggtttttaaaaagattgctgaacatggaccagaatataggtttgagaagttaggagagatgataatggcttcgaattatgcaggtattgcatttggaaatgctggtgttggtgctgtacatgcattatcttaccctttaggtggaaattatcatgttccacatggagaagcaaactatcaattttttaccgaagtttttaaagtttatcaaaagaaaaatccgtttggatatatcgttgagttaaactggaaactttccaaaattctcaattgtcaacctgaatacgtatatcctaagctagatgaacttctcggttgtcttttgaccaaaaaacccttacatgaatatggaatgaaagatgaagaagttagaggatttgctgaatctgtgttaaagacgcaacaaagattattggctaacaattatgtcgaacttacagtagatgagatagaagggatttataggagactttactagagcttccgcggaggccggcgcggccgatttaaatcctcagcctgagcccccgggcggtatcagctcactcaaaggcggtaataacaactttgtatacaaaagttgtagtgtagtagcctgtgaaataagtaaggaaaaaaaagaagtaagtgttatatatgatgattattttgtagatgtagataggataatagaatccatagaaaatataggttatacagttatataaaaattactttaaaaattaataaaaacatggtaaaatataaatcgtataaagttgtgtaatttttaaggaggtgtgttacatatgcaatggcaagaactttaccgtcagcgcgtttgctctgcagacgaagctgtcgtggactctcttaaaccgggaacgaaagttgtattcggtcatgctgctgctgcgcctgtccgtttctctcaggctatgtaccgccagcgtgaaaagttggagaatatcacagttttccacatgttgtatttcggcgacgcgccgcaccttgctcccgaaatgcgttcgcatgtacacccgactctcaacttccttgagggcaactcccgtccggcaagccgtgaccgtcgtgtcgatttcattccctgccacttccacgaggtaccggaactgtttcgtcagggattctttccattggatgtagccgtagtgcaggtatctactcctaacgaagagggttattgctctttcggagtttcctgcgactacacaaaggctgccgccgagtgcgctccggtagtagtagccgaggtgaacaagcaaatgccattcatcggtggtgaaaacctgattcacatctccaaactgacccatatcatcgaagtggacgagccgattgcagaagtattgcctcctgctatcagcgaccttgaactgaggataggtcagaattgtgcctcactgatcaaagacggcgataccctccagttgggtatcggcggtatccccgacgctgtgttgcgtgcattggaagggcataaagatctcggtattcacacggaaatgtttaccgacggggtgatgcgtatgattcgcaaggggattatcaacgggaagaaaaaaacattgcatcccgaaaaagtcgttacctcgctaatcttcggatcgaaagaattgtacgattttgtcaataacaatccggtgatagaatgctatccggtggattatatcaacaaccccgatgttatcggtaagaatgaccgcatggtttctatcaattcctgcttggagatggatctcatggggcaggcagcttctgagtcgatcgggtacgaacagttcagtggatccggaggtcaagtcgatttccttcgtggggccaagcgttccaagggaggaatctccattatggctttccccagtacggccaagaaagggactgagagtcgcatcgttcccattctgaaagagggtgcttgtgtcacgaccggccgtaacgaagtggactatgtggtgacggaatatggcgtagcgcgtctgcgtggcgcaacgcttcgtcagcgtgctgaagccttgactgctatagcacatcccgatttccgaccggcccttgaggaggaaatccgccgacgcttcgaataagcatgcacggtagccaggaggttaatgaaagttacaaatcaaaaagaactaaaacaaaagctaaatgaattgagagaagcgcaaaagaagtttgcaacctatactcaagagcaagttgataaaatttttaaacaatgtgccatagccgcagctaaagaaagaataaacttagctaaattagcagtagaagaaacaggaataggtcttgtagaagataaaattataaaaaatcattttgcagcagaatatatatacaataaatataaaaatgaaaaaacttgtggcataatagaccatgacgattctttaggcataacaaaggttgctgaaccaattggaattgttgcagccatagttcctactactaatccaacttccacagcaattttcaaatcattaatttctttaaaaacaagaaacgcaatattcttttcaccacatccacgtgcaaaaaaatctacaattgctgcagcaaaattaattttagatgcagctgttaaagcaggagcacctaaaaatataataggctggatagatgagccatcaatagaactttctcaagatttgatgagtgaagctgatataatattagcaacaggaggtccttcaatggttaaagcggcctattcatctggaaaacctgcaattggtgttggagcaggaaatacaccagcaataatagatgagagtgcagatatagatatggcagtaagctccataattttatcaaagacttatgacaatggagtaatatgcgcttctgaacaatcaatattagttatgaattcaatatacgaaaaagttaaagaggaatttgtaaaacgaggatcatatatactcaatcaaaatgaaatagctaaaataaaagaaactatgtttaaaaatggagctattaatgctgacatagttggaaaatctgcttatataattgctaaaatggcaggaattgaagttcctcaaactacaaagatacttataggcgaagtacaatctgttgaaaaaagcgagctgttctcacatgaaaaactatcaccagtacttgcaatgtataaagttaaggattttgatgaagctctaaaaaaggcacaaaggctaatagaattaggtggaagtggacacacgtcatctttatatatagattcacaaaacaataaggataaagttaaagaatttggattagcaatgaaaacttcaaggacatttattaacatgccttcttcacagggagcaagcggagatttatacaattttgcgatagcaccatcatttactcttggatgcggcacttggggaggaaactctgtatcgcaaaatgtagagcctaaacatttattaaatattaaaagtgttgctgaaagaagggaaaatatgctttggtttaaagtgccacaaaaaatatattttaaatatggatgtcttagatttgcattaaaagaattaaaagatatgaataagaaaagagcctttatagtaacagataaagatctttttaaacttggatatgttaataaaataacaaaggtactagatgagatagatattaaatacagtatatttacagatattaaatctgatccaactattgattcagtaaaaaaaggtgctaaagaaatgcttaactttgaacctgatactataatctctattggtggtggatcgccaatggatgcagcaaaggttatgcacttgttatatgaatatccagaagcagaaattgaaaatctagctataaactttatggatataagaaagagaatatgcaatttccctaaattaggtacaaaggcgatttcagtagctattcctacaactgctggtaccggttcagaggcaacaccttttgcagttataactaatgatgaaacaggaatgaaataccctttaacttcttatgaattgaccccaaacatggcaataatagatactgaattaatgttaaatatgcctagaaaattaacagcagcaactggaatagatgcattagttcatgctatagaagcatatgtttcggttatggctacggattatactgatgaattagccttaagagcaataaaaatgatatttaaatatttgcctagagcctataaaaatgggactaacgacattgaagcaagagaaaaaatggcacatgcctctaatattgcggggatggcatttgcaaatgctttcttaggtgtatgccattcaatggctcataaacttggggcaatgcatcacgttccacatggaattgcttgtgctgtattaatagaagaagttattaaatataacgctacagactgtccaacaaagcaaacagcattccctcaatataaatctcctaatgctaagagaaaatatgctgaaattgcagagtatttgaatttaaagggtactagcgataccgaaaaggtaacagccttaatagaagctatttcaaagttaaagatagatttgagtattccacaaaatataagtgccgctggaataaataaaaaagatttttataatacgctagataaaatgtcagagcttgcttttgatgaccaatgtacaacagctaatcctaggtatccacttataagtgaacttaaggatatctatataaaatcattttaacccgggaacccagctttcttgtacaaagtggtgatgatccggctgctaacctcgagaacatgtgagcaataaaaataagaagcctgcatttgcaggcttcttatttttatagaccggggacttatcagccaacctgtaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacggtcctgcaggtaatacgactcactatagggataacttaaatttatattttttactttataatatataattgattatagaatccaaattagataggaggaccaggtaaatggagaataattttattgtcaacgaaaacgagaaaagggttttaaagcagatatttaacaactccaatatcagtagaactcaaatttccaagaatttagaactgaataaagcaacaatttcgaacattcttaacaatctaaagcataaaagtttggtaaatgaagttggtgaaggaaattctactaaaagtggtggaagaaaacctatacttctcgaaattaatcaaaagtatgggtattatataagcatggatttgacatatgattctgttgagttaatgtataactattttgatgctacaattctcaaacaagatagttatgaactgaatgataagaatgtaagtagcatattacagatactcaaatctaacataaatgttagcgaaaaatacgacacattatacggtttattagggataagtatatccatacatgggattgtagatgatgaacaaaacatcattaatctaccatttcacaagaatgaaaaaagaacgtttactgatgaacttaagtctttcactaatgtaccagtggttattgaaaatgaggcaaatttaagtgcattatacgagaaaagtttatacatcaattcgaatattaacaacttgatcacattgtctatacataaaggtataggagctggcatcataatcaataagaaactctatagaggtagtaatggagaagctggagaaataggaaagactcttgttttagagtctatcaataacaatgataacaagtactataaaatagaggatatttgtagtcaagacgctttgatacaaaagattaataacagattaggagttacacttacatttaccgaactaatacagtattataacgaaggaaatagtattgtagctcatgaaatcaaacagtttatcaacaaaatgactgtcttaatacacaatcttaatactcagtttaatccagatgcaatatacatcaattgtcccttgattaatgaacttcctaatattctaaacgaaatcaaagaacaattctcttgtttttcacaaggaagtcctatacaattgcatttaaccactaatgttaaacaagctacattattaggtggaacattagcaattatgcagaaaacccttaacataaacaatatccaaatgaatattaaatagttacagcagtctgagttataaaatagatatctcggaccgtctattcttccttatttgagtgggtaccaatgaaatctttcgagctaaatcgatttctggcccactcttttttattttttagtaagcgtttacaaaaaatgaacaatgtgctatattacctctaaagttagtttgtttattaaattaaccaactaaaatgtcgggatccaattaggaggaatctgcgatggaaaagaaggaatttagagttttaataaagtattgttttcttaaaggaaagaatacagtagaagctaagacttggttagataatgaatttccagattctgctcctggtaaaagtacaattatagattggtatgcaaaatttaaaagaggagaaatgtcaacagaagatggagaaagatctggtagaccaaaagaagttgtaactgatgaaaatataaagaagatacataaaatgatattaaatgatagaaagatgaagttaatagaaatagcagaagctttaaaaatatctaaagaaagagttggacatataatacatcaatatttagatatgagaaaattatgtgcaaaatgggttccaagagaattaacttttgatcaaaagcaaagaagagtagatgatagtaagagatgtttacaattattaacaagaaatactccagaatttttcagaagatatgtaacaatggatgaaacttggttacatcattatacacctgaaagtaatagacaatcagcagaatggacagctactggagaaccatcacctaaaagaggaaagactcaaaagagtgctggaaaggttatggcatcagtattttgggatgctcatggtataatatttatagattatttagaaaaaggaaaaacaataaattctgattattatatggcattattagaaagattaaaagttgaaatagcagctaaaagaccacatatgaagaagaaaaaagtattatttcatcaagataatgctccttgtcataagtcattaagaactatggcaaagatacatgaattaggttttgaattattaccacatccaccttattcaccagatttagctccttctgatttctttttatttagtgatttaaagagaatgttagcaggaaagaagtttggttgtaatgaagaagttatagcagaaacagaagcttattttgaagcaaaacctaaggaatattatcaaaatggaataaagaagttagaaggtagatataatagatgtatagcattagaaggaaattatgtagaataagaattccttaattaaggagctcgataaaaataagaagcctgcatttgcaggcttcttatttttatggtcatagctgtttcctggcgcgccgcattcacttcttttctatataaatatgagcgaagcgaataagcgtcggaaaagcagcaaaaagtttcctttttgctgttggagcatgggggttcagggggtgcagtatctgacgtcaatgccgagcgaaagcgagccgaagggtagcatttacgttagataaccccctgatatgctccgacgctttatatagaaaagaagattcaactaggtaaaatcttaatataggttgagatgataaggtttataaggaatttgtttgttctaatttttcactcattttgttctaatttcttttaacaaatgttcttttttttttagaacagttatgatatagttagaatagtttaaaataaggagtgagaaaaagatgaaagaaagatatggaacagtctataaaggctctcagaggctcatagacgaagaaagtggagaagtcatagaggtagacaagttataccgtaaacaaacgtctggtaacttcgtaaaggcatatatagtgcaattaataagtatgttagatatgattggcggaaaaaaacttaaaatcgttaactatatcctagataatgtccacttaagtaacaatacaatgatagctacaacaagagaaatagcaaaagctacaggaacaagtctacaaacagtaataacaacacttaaaatcttagaagaaggaaatattataaaaagaaaaactggagtattaatgttaaaccctgaactactaatgagaggcgacgaccaaaaacaaaaatacctcttactcgaatttgggaactttgagcaagaggcaaatgaaatagattgacctcccaataacaccacgtagttattgggaggtcaatctatgaaatgcgattaagggccggccagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaacaagtatttaagcaaaaacatcgtagaaatacggtgttttttgttaccctaagttt

SEQ ID NO: 5 - DNA, 6774 bp, Artificial
  Synthetic gene cluster for 1,4-BDO synthesis in Clostridium ljung dahlii
agatctatgagtaatgaagtatctataaaagaattaattgaaaaggcaaaggtggcacaaaaaaaattggaagcctatagtcaagaacaagttgatgtactagtaaaagcactaggaaaagtggtttatgataatgcagaaatgtttgcaaaagaagcagttgaagaaacagaaatgggtgtttatgaagataaagtagctaaatgtcatttgaaatcaggagctatttggaatcatataaaagacaagaaaactgtaggcataataaaagaagaacctgaaagggcacttgtttatgttgctaagccaaagggagttgtggcagctactacgcctataactaatccagtggtaactcctatgtgtaatgcaatggctgctataaagggcagaaatacaataatagtagcaccacatcctaaagcaaagaaagtttcagctcatactgtagaacttatgaatgctgagcttaaaaaattgggagcaccagaaaatatcatacagatagtagaagcaccatcaagagaagctgctaaggaacttatggaaagtgctgatgtagttattgctacaggcggtgctggaagagttaaagctgcttactccagtggaagaccagcttatggcgttggacctggaaattcacaggtaatagttgataagggatacgattataacaaagctgcacaggatataataacaggaagaaaatatgacaatggaattatatgttcttcagagcaatcagttatagctcctgctgaagattatgataaggtaatagcagcttttgtagaaaatggggcattctatgtagaagatgaggaaacagtagaaaagtttagatcaactttatttaaagatggaaaaataaacagcaagattataggtaaatccgtccaaattattgcggatcttgcaggagtaaaagtaccagaaggtactaaggttatagtacttaagggtaaaggtgcaggagaaaaagatgtactttgtaaagaaaaaatgtgtccagttttagtagcattgaaatatgatacttttgaagaagcagttgaaatagctatggctaattatatgtatgaaggagctggtcatacagcaggcatacattctgacaatgacgagaacataagatatgcaggaactgtattacctataagcagattagttgtaaatcagcctgcaactactgctggaggaagtttcaataatggatttaaccctactactacactaggctgcggatcatggggcagaaacagtatttcagaaaatcttacttacgagcatcttataaatgtttcaagaatagggtatttcaataaagaagcaaaagttcctagctatgaggaaatatggggataataattcttaaataaacaatacttaaaacatttgaggaggtcttgtaaacatgcaacttttcaaactcaagagtgtaacacatcactttgacacttttgcagaatttgccaaggaattctgtcttggagaacgcgacttggtaattaccaacgagttcatctatgaaccgtatatgaaggcatgccagctcccctgccattttgttatgcaggagaaatatgggcaaggcgagccttctgacgaaatgatgaataacatcttggcagacatccgtaatatccagttcgaccgcgtaatcggtatcggaggaggtacggttattgacatctctaaacttttcgttctgaaaggattaaatgatgtactcgatgcattcgaccgcaaaatacctcttatcaaagagaaagaactgatcattgtgcccacaacatgcggaacgggtagcgaggtgacgaacatttctatcgcagaaatcaaaagccgtcacaccaaaatgggattggctgacgatgccattgttgcagaccatgccatcatcatacctgaacttctgaagagcttgcctttccacttctacgcatgcagtgcaatcgatgctcttatccatgccatcgagtcatacgtatctcctaaagccagtccatattctcgtctgttcagtgaggcggcttgggacattatcctggaagtattcaagaaaatcgccgaacacggccctgaataccgcttcgaaaagctgggagaaatgatcatggccagcaactatgccggtatagccttcggaaatgcaggagtaggagccgtccacgcactatcctacccgttgggaggcaactatcacgtgccgcatggagaagcaaactatcagttcttcacagaggtattcaaagtataccaaaagaagaatcctttcggctatatagtcgaactcaactggaagctctccaagatactgaactgccagcccgaatacgtatatccgaagctggatgaacttctcggatgccttcttaccaagaaacctttgcacgaatacggcatgaaggacgaagaggtaagaggctttgcggaatcagtgcttaagacacagcaaagattgctcgccaacaactacgtagagcttactgtagatgagatcgaaggtatctacagaagactctactaataatagcataaccccttggggcctctaaacgggtcttgaggggttttttggcgtgtgtgttagccaaagctcctgcaggtcgactttttaacaaaatatattgataaaaataataatagtgggtataattaagttgttagagaaaacgtataaattagggataaactatggaacttatgaaatagattgaaatggtttatctgttaccccgtaggatccagaatttaaaaggagggattaaaatgaaagttacaaatcaaaaagaactaaaacaaaagctaaatgaattgagagaagcgcaaaagaagtttgcaacctatactcaagagcaagttgataaaatttttaaacaatgtgccatagccgcagctaaagaaagaataaacttagctaaattagcagtagaagaaacaggaataggtcttgtagaagataaaattataaaaaatcattttgcagcagaatatatatacaataaatataaaaatgaaaaaacttgtggcataatagaccatgacgattctttaggcataacaaaggttgctgaaccaattggaattgttgcagccatagttcctactactaatccaacttccacagcaattttcaaatcattaatttctttaaaaacaagaaacgcaatattcttttcaccacatccacgtgcaaaaaaatctacaattgctgcagcaaaattaattttagatgcagctgttaaagcaggagcacctaaaaatataataggctggatagatgagccatcaatagaactttctcaagatttgatgagtgaagctgatataatattagcaacaggaggtccttcaatggttaaagcggcctattcatctggaaaacctgcaattggtgttggagcaggaaatacaccagcaataatagatgagagtgcagatatagatatggcagtaagctccataattttatcaaagacttatgacaatggagtaatatgcgcttctgaacaatcaatattagttatgaattcaatatacgaaaaagttaaagaggaatttgtaaaacgaggatcatatatactcaatcaaaatgaaatagctaaaataaaagaaactatgtttaaaaatggagctattaatgctgacatagttggaaaatctgcttatataattgctaaaatggcaggaattgaagttcctcaaactacaaagatacttataggcgaagtacaatctgttgaaaaaagcgagctgttctcacatgaaaaactatcaccagtacttgcaatgtataaagttaaggattttgatgaagctctaaaaaaggcacaaaggctaatagaattaggtggaagtggacacacgtcatctttatatatagattcacaaaacaataaggataaagttaaagaatttggattagcaatgaaaacttcaaggacatttattaacatgccttcttcacagggagcaagcggagatttatacaattttgcgatagcaccatcatttactcttggatgcggcacttggggaggaaactctgtatcgcaaaatgtagagcctaaacatttattaaatattaaaagtgttgctgaaagaagggaaaatatgctttggtttaaagtgccacaaaaaatatattttaaatatggatgtcttagatttgcattaaaagaattaaaagatatgaataagaaaagagcctttatagtaacagataaagatctttttaaacttggatatgttaataaaataacaaaggtactagatgagatagatattaaatacagtatatttacagatattaaatctgatccaactattgattcagtaaaaaaaggtgctaaagaaatgcttaactttgaacctgatactataatctctattggtggtggatcgccaatggatgcagcaaaggttatgcacttgttatatgaatatccagaagcagaaattgaaaatctagctataaactttatggatataagaaagagaatatgcaatttccctaaattaggtacaaaggcgatttcagtagctattcctacaactgctggtaccggttcagaggcaacaccttttgcagttataactaatgatgaaacaggaatgaaataccctttaacttcttatgaattgaccccaaacatggcaataatagatactgaattaatgttaaatatgcctagaaaattaacagcagcaactggaatagatgcattagttcatgctatagaagcatatgtttcggttatggctacggattatactgatgaattagccttaagagcaataaaaatgatatttaaatatttgcctagagcctataaaaatgggactaacgacattgaagcaagagaaaaaatggcacatgcctctaatattgcggggatggcatttgcaaatgctttcttaggtgtatgccattcaatggctcataaacttggggcaatgcatcacgttccacatggaattgcttgtgctgtattaatagaagaagttattaaatataacgctacagactgtccaacaaagcaaacagcattccctcaatataaatctcctaatgctaagagaaaatatgctgaaattgcagagtatttgaatttaaagggtactagcgataccgaaaaggtaacagccttaatagaagctatttcaaagttaaagatagatttgagtattccacaaaatataagtgccgctggaataaataaaaaagatttttataatacgctagataaaatgtcagagcttgcttttgatgaccaatgtacaacagctaatcctaggtatccacttataagtgaacttaaggatatctatataaaatcattttaataattcttaaataaacaatacttaaaacatttgaggaggtcttgtaaacatggattggaagaagatctatgaagacagaacatgcactgcagatgaagcagtaaagagcattaagtcaggtgacagagtgctatttgcgcactgtgttgctgaaccgccagttcttgtagaagcaatggttgcgaatgcagctgcatacaagaatgtaacggtttcacacatggttacccttggaaagggtgaatactcaaaaccagaatataaggaaaactttacttttgaaggttggtttacaagcccttcaacaagaggatccattgcagaaggacacggacagtttgtccctgtattcttccacgaggtaccatctttaatcagaaaagacattttccatgttgatgtattcatggtaatggtatcccctccagatcataacggattctgctgtgtgggtgtatcttctgactatacgatgcaggctatcaaatcagcaaaaattgtacttgctgaagtaaatgatcaggtacctgtagtttatggagatacatttgttcacgttagtgaaatcgacaagttcgtagaaacttcacatccacttccagaaatcggacttcctaagatcggtgaagtagaagctgctattggtaagcactgcgcttccctaatcgaagatggttccacattacagcttggtatcggagctattccggatgctgtactttcacagcttaaggacaagaaacaccttggtatccactctgaaatgatttccgacggtgttgtagatctttacgaagcaggcgttatagactgcagccaaaagtctatcgacaaaggcaaaatggcaataacattcttaatgggaacgaagagactttatgatttcgctgcaaacaatccaaaggttgaattaaagccggttgactacataaatcatccatctgtagttgcacagtgctccaaaatggtttgcatcaatgcttgcttgcaagttgattttatgggtcagattgtatccgatagtattgggacaaagcagttctcaggagtaggcggtcaggttgacttcgtaagaggtgcatccatgtctattgacggaaaaggtaaagcgatcatcgcgatgccttccgttgcaaagaagaaggatggaagtatgatttcgaagatcgttccattcatcgatcacggtgcagctgtaactacatccagaaacgatgcggactatgtcgtaacggaatatggtattgctgaaatgaagggtaagtctttacaggacagagcaagagcgttaatcaatattgcacaccctgatttcaaagatgaattaaaggctgaatttgaaaagagattcaacgcggcattctaataatagcataaccccttggggcctctaaacgggtcttgaggggttttttgctcgag

SEQ ID NO: 6 - DNA, 120 bp, Artificial
  Linker
ggatccgcgtcccccgggggactagctagctaggaattcgagctcggcgcgcctgcaggtcgacaagcttgcggccgcataatgcttaagccgctcgagcggccttaattaaggggcgcc

(19) United States
(12) Patent Application Publication
(10) Pub. No.: US 2018/0340194 A1
(43) Pub. Date: Nov. 29, 2018

(54) RECOMBINANT CELLS, METHOD FOR PRODUCING RECOMBINANT CELLS, AND METHOD FOR PRODUCING 1,4-BUTANEDIOL

(71) Applicants: SEKISUI CHEMICAL CO., LTD., Osaka (JP); FRAUNHOFER-GESELLSCHAFT ZUR FÖRDERUNG DER ANGEWANDTEN FORSCHUNG E.V., München (DE)

(72) Inventors: Masahiro FURUTANI, Arakawa-ku, Tokyo (JP); Stefan JENNEWEIN, Wichfriedweg, Bad Honnef (DE); Rainer FISCHER, Indianapolis, IN (US); Christopher MCELROY, Hubertusstrase, Aachen (DE); Stefan GAIDA, Hans-Thoma-Str., Potsdam (DE)

(73) Assignees: SEKISUI CHEMICAL CO., LTD., Osaka (JP); FRAUNHOFER-GESELLSCHAFT ZUR FÖRDERUNG DER ANGEWANDTEN FORSCHUNG E.V., München (DE)

(21) Appl. No.: 15/755,808

(22) PCT Filed: Sep. 2, 2015
(86) PCT No.: PCT/JP2015/074979

§ 371 Date: Feb. 27, 2018

(51) Int. Cl. C12P 7/18; C12N 1/20; C12N 15/00

(52) U.S. Cl. CPC C12P 7/18; C12N 15/00; C12N 1/20

(57) ABSTRACT

An object of the present invention is to provide a technique for producing 1,4-butanediol by recombinant cells. Provided is a recombinant cell that is acetogenic and obligatory anaerobic, wherein the recombinant cell includes a gene encoding at least one enzyme selected from the group consisting of succinate semialdehyde dehydrogenase, succinyl-CoA synthase, CoA-dependent succinate semialdehyde dehydrogenase, 4-hydroxybutyrate dehydrogenase, 4-hydroxybutyryl-CoA transferase, 4-hydroxybutyryl-CoA reductase, 4-hydroxybutyraldehyde dehydrogenase, and alcohol dehydrogenase, the gene is expressed in the recombinant cell, and the recombinant cell produces 1,4-butandiol.

### TECHNICAL FIELD

The present invention relates to a recombinant cell, a method for producing a recombinant cell, and a method for producing 1,4-butanediol.

### BACKGROUND ART

1,4-Butanediol is an organic compound that can be a raw material of butadiene which is important as a monomer of synthetic rubber, and is an important material, in particular, in the tire industry. In recent years, the technique for conversion from a production process of basic chemicals relying on petroleum to a production process from renewable resources such as plant resources has been developed and practical realization thereof is steadily progressing. Also regarding 1,4-butanediol, for example, a production technique from saccharides as a raw material by recombinant Escherichia coli is known (Patent Document 1).

FIG. 1 shows an example of the biosynthesis pathway of 1,4-butanediol. Specifically, 1,4-butanediol can be biosynthesized, for example, from succinate or ca-ketoglutarate as a starting material.

In the pathway starting from succinate, succinate is converted into 1,4-butanediol via succinyl CoA, succinyl semialdehyde, 4-hydroxybutyrate, 4-hydroxybutyryl CoA, and 4-hydroxybutyraldehyde. Enzymes that catalyze these reactions are (a) succinyl-CoA synthase, (b) CoA-dependent succinate semialdehyde dehydrogenase, (e) 4-hydroxybutyrate dehydrogenase, (f) 4-hydroxybutyryl-CoA transferase, (g) 4-hydroxybutyryl-CoA reductase, and (h) alcohol dehydrogenase, respectively (FIG. 1). Every organism has (a) succinyl-CoA synthase.

Also, there is a pathway that directly converts succinate into succinyl semialdehyde. In that case, succinate is converted into 1,4-butanediol via succinyl semialdehyde, 4-hydroxybutyrate, 4-hydroxybutyryl CoA, and 4-hydroxybutyraldehyde. The enzyme that catalyzes the reaction of converting succinate into succinyl semialdehyde is (c) succinate semialdehyde dehydrogenase (FIG. 1).

On the other hand, in the pathway starting from α-ketoglutarate, α-ketoglutarate is converted into 1,4-butanediol via succinyl semialdehyde, 4-hydroxybutyrate, 4-hydroxybutyryl CoA, and 4-hydroxybutyraldehyde. The enzyme that catalyzes the reaction of converting α-ketoglutarate into succinyl semialdehyde is (d) 2-oxoglutarate decarboxylase (FIG. 1).

Further, there is a pathway that generates 4-hydroxybutyraldehyde directly from 4-hydroxybutyrate by (i) 4-hydroxybutyraldehyde dehydrogenase (FIG. 1).

Regarding the production process from renewable resources, most of the conventional techniques including the aforementioned 1,4-butanediol production technique are production methods by microorganisms relying on organic substances, in particular, saccharides, glycerol or oil components. However, for covering the global production quantity of a large number of basic chemicals derived from petroleum, the amounts of currently available saccharides, glycerin and oil components derived from plant resources and the like will be necessarily insufficient for carbon sources of microorganisms. In other words, the production amounts of basic chemicals by microorganisms relying on saccharides or oil components is limited also in the future. These processes also have a fear of competition with foods.

Syngas (synthesis gas) is a mixed gas mainly containing carbon monoxide, carbon dioxide, and hydrogen, which is efficiently obtained by burning waste, natural gas and coal. In the field of C1 chemistry by metal catalysts starting from syngas, a process for mass production of liquid chemicals such as methanol, formic acid and formaldehyde at low costs has been developed.

Carbon monoxide, carbon dioxide and hydrogen are contained in syngas derived from waste, industrial exhaust gas, natural gas or syngas derived from coal, and are available almost permanently. However, at present, examples of producing chemicals by microorganisms from C1 carbon sources represented by syngas are very limited. Only production of ethanol, 2,3-butanediol or the like from syngas is currently under development. In particular, there is little report about utilization of a syngas utilizing substance by a recombinant. Patent Document 2 discloses a production technique of isopropanol by a recombinant of Escherichia coli. In this technique, a plurality of CO metabolic enzyme genes are introduced into Escherichia coli to impart a syngas utilizing ability, and isopropanol is produced from syngas. However, this technique does not relate to production of 1,4-butanediol. In addition, high productivity of an intended substance cannot be expected from a system using recombinant Escherichia coli having CO metabolism, due to reasons such as the difficultly of functionally and efficiently expressing in Escherichia coli a group of CO metabolic enzyme genes held originally by a syngas assimilating microorganism, and CO tolerance of Escherichia coli being low.

Lignocellulose is the most abundantly available non-food-based biomass. Lignocellulose can break down into cellulose, hemicellulose and lignin by conducting a physical or chemical treatment. Specifically, cellulose is expected to be a carbon source available for culturing a microorganism. However, to utilize cellulose efficiently, it is necessary to saccharify cellulose in advance by physical, chemical and enzymatic reactions, and the cost is high. Therefore, technology to cheaply saccharify cellulose and to further convert it into energies or useful materials is desired. Technology to effectively produce 1,4-butanediol from cellulose has not been found until now.

### PRIOR ART DOCUMENTS

### Patent Document

Patent Document 1: JP 2012-529267 A
Patent Document 2: JP 2012-509691 A

### DISCLOSURE OF INVENTION

### Technical Problem

In light of the above, it is an object of the present invention to provide a series of techniques capable of producing 1,4-butanediol from cellulose or syngas which does not either rely on petroleum or compete with foods.

### Solution to Problem

One aspect of the present invention for solving the aforementioned problem is a recombinant cell that is acetogenic and obligatory anaerobic, wherein the recombinant cell includes a gene encoding at least one enzyme selected from the group consisting of succinate semialdehyde dehydrogenase, succinyl-CoA synthase, CoA-dependent succinate semialdehyde dehydrogenase, 4-hydroxybutyrate dehydrogenase, 4-hydroxybutyryl-CoA transferase, 4-hydroxybutyryl-CoA reductase, 4-hydroxybutyraldehyde dehydrogenase, and alcohol dehydrogenase, the gene is expressed in the recombinant cell, and the recombinant cell produces 1,4-butandiol.

As illustrated in FIG. 1, 1,4-butanediol can be biosynthesized from succinate. The recombinant cell of the present invention, which is acetogenic and obligatory anaerobic, includes a gene encoding at least one enzyme selected from the group of enzyme acting in a biosynthesis pathway from succinate to 1,4-butanediol, namely the group consisting of succinate semialdehyde dehydrogenase, succinyl-CoA synthase, CoA-dependent succinate semialdehyde dehydrogenase, 4-hydroxybutyrate dehydrogenase, 4-hydroxybutyryl-CoA transferase, 4-hydroxybutyryl-CoA reductase, 4-hydroxybutyraldehyde dehydrogenase, and alcohol dehydrogenase. The gene is expressed in the recombinant cell, and the recombinant cell produces 1,4-butandiol from at least one C1 compound selected from the group consisting of carbon monoxide, carbon dioxide, formic acid, and methanol. According to the recombinant cell of the present invention, it is possible to produce 1,4-butanediol from, for example, cellulose or syngas that contains carbon monoxide and/or carbon dioxide.

Another aspect of the present invention for solving a similar problem is a recombinant cell that is acetogenic and obligatory anaerobic, wherein the recombinant cell includes a gene encoding at least one enzyme selected from the group consisting of 2-oxoglutarate decarboxylase, 4-hydroxybutyrate dehydrogenase, 4-hydroxybutyryl-CoA transferase, 4-hydroxybutyryl-CoA reductase, 4-hydroxybutyraldehyde dehydrogenase, and alcohol dehydrogenase, the gene is expressed in the recombinant cell, and the recombinant cell produces 1,4-butandiol.

As illustrated in FIG. 1, 1,4-butanediol can be biosynthesized also from α-ketoglutarate. The recombinant cell of the present invention, which is acetogenic and obligatory anaerobic, includes a gene encoding at least one enzyme selected from the group of enzyme acting in a biosynthesis pathway from α-ketoglutarate to 1,4-butanediol, namely the group consisting of 2-oxoglutarate decarboxylase, 4-hydroxybutyrate dehydrogenase, 4-hydroxybutyryl-CoA transferase, 4-hydroxybutyryl-CoA reductase, 4-hydroxybutyraldehyde dehydrogenase, and alcohol dehydrogenase. The gene is expressed in the recombinant cell, and the recombinant cell produces 1,4-butandiol from at least one C1 compound selected from the group consisting of carbon monoxide, carbon dioxide, formic acid, and methanol. According to the recombinant cell of the present invention, it is possible to produce 1,4-butanediol from, for example, cellulose or syngas that contains carbon monoxide and/or carbon dioxide.

Preferably, the recombinant cell has a function of synthesizing acetyl-CoA from methyltetrahydrofolate, carbon monoxide, and CoA, and the recombinant cell produces 1,4-butandiol from at least one C1 compound selected from the group consisting of carbon monoxide, carbon dioxide, formic acid, and methanol.

With such a constitution, 1,4-butanediol can be produced from the C1 compound via succinate or α-ketoglutarate based on the “function of synthesizing acetyl-CoA from methyltetrahydrofolate, carbon monoxide, and CoA”, which is included in the recombinant cell.

Examples of the cell having a “function of synthesizing acetyl-CoA from methyltetrahydrofolate, carbon monoxide, and CoA” include anaerobic microorganisms having an acetyl-CoA pathway (Wood-Ljungdahl pathway) and a methanol pathway shown in FIG. 2.

Preferably, the recombinant cell has carbon monoxide dehydrogenase.

Carbon monoxide dehydrogenase (EC 1.2.99.2/1.2.7.4) (CO dehydrogenase, CODH) has an activity of generating carbon dioxide and proton from carbon monoxide and water, and an activity of generating carbon monoxide and water from carbon dioxide and proton, which is a reverse reaction. The carbon monoxide dehydrogenase is one of enzymes that act in the acetyl-CoA pathway (FIG. 2).

Preferably, the recombinant cell is a Clostridium bacterium, a Moorella bacterium, or an Acetobacterium bacterium.

Preferably, the recombinant cell has cellulose assimilating ability, and the recombinant cell produces 1,4-butandiol from lignocellulose, cellulose, or soluble carbohydrate.

With such a constitution, 1,4-butanediol can be produced from lignocellulose, cellulose, or soluble carbohydrate via succinate or α-ketoglutarate based on the cellulose assimilating ability included in the recombinant cell, specifically on the function of synthesizing acetyl-CoA from cellulose. The pathway of synthesizing acetyl-CoA from cellulose mainly consists of Embden-Meyerhof-Parnas pathway.

Preferably, the recombinant cell produces cellulosome.

Preferably, the recombinant cell is a Clostridium bacterium.

Preferably, the recombinant cell is Clostridium thermocellum, Clostridium cellulolyticum, or Clostridium cellulovorans.

Preferably, at least one of the genes is derived from a bacterium.

Preferably, at least one of the genes is derived from its original host cell.

Preferably, the gene is incorporated in a genome of the host cell.

With such a configuration, the gene is retained in the recombinant cell more stably.

Preferably, the recombinant cell is tolerant to at least 400 mM 1,4-butanediol.

With such a configuration, a larger amount of 1,4-butanediol can be produced.

Preferably, ethanol generating ability of the recombinant cell is down-regulated compared to that of its original host cell.

Preferably, 2,3-butanediol generating ability of the recombinant cell is down-regulated compared to that of its original host cell.

Another aspect of the present invention is a method for producing a recombinant cell, including: a first step of providing a host cell that is acetogenic and obligatory anaerobic; and a second step of introducing a gene encoding at least one enzyme selected from the group consisting of succinate semialdehyde dehydrogenase, succinyl-CoA synthase, CoA-dependent succinate semialdehyde dehydrogenase, 4-hydroxybutyrate dehydrogenase, 4-hydroxybutyryl-CoA transferase, 4-hydroxybutyryl-CoA reductase, 4-hydroxybutyraldehyde dehydrogenase, and alcohol dehydrogenase, into the host cell, wherein the gene is expressed in the recombinant cell, and the recombinant cell produces 1,4-butandiol.

Another aspect of the present invention is a method for producing a recombinant cell, including: a first step of providing a host cell that is acetogenic and obligatory anaerobic; and a second step of introducing a gene encoding at least one enzyme selected from the group consisting of 2-oxoglutarate decarboxylase, 4-hydroxybutyrate dehydrogenase, 4-hydroxybutyryl-CoA transferase, 4-hydroxybutyryl-CoA reductase, 4-hydroxybutyraldehyde dehydrogenase, and alcohol dehydrogenase, into the host cell, wherein the gene is expressed in the recombinant cell, and the recombinant cell produces 1,4-butandiol.

Preferably, the host cell has a function of synthesizing acetyl-CoA from methyltetrahydrofolate, carbon monoxide, and CoA, and the recombinant cell produces 1,4-butandiol from at least one C1 compound selected from the group consisting of carbon monoxide, carbon dioxide, formic acid, and methanol.

Preferably, the host cell has carbon monoxide dehydrogenase.

Preferably, the host cell is a Clostridium bacterium, a Moorella bacterium, or an Acetobacterium bacterium.

Preferably, the host cell has cellulose assimilating ability, and the recombinant cell produces 1,4-butandiol from lignocellulose, cellulose, or soluble carbohydrate.

Preferably, the host cell produces cellulosome.

Preferably, the host cell is a Clostridium bacterium.

Preferably, the host cell is Clostridium thermocellum, Clostridium cellulolyticum, or Clostridium cellulovorans.

Preferably, at least one of the genes is derived from a bacterium.

Preferably, at least one of the genes is derived from the host cell.

Preferably, wherein the gene introduced in the second step is incorporated in a genome.

Preferably, the recombinant cell is tolerant to at least 400 mM 1,4-butanediol.

Preferably, ethanol generating ability of the recombinant cell is down-regulated compared to that of the host cell.

Preferably, 2,3-butanediol generating ability of the recombinant cell is down-regulated compared to that of the host cell.

Another aspect of the present invention is a method for producing 1,4-butanediol, including: bringing at least one C1 compound selected from the group consisting of carbon monoxide, carbon dioxide, formic acid, and methanol into contact with the aforementioned recombinant cell or the recombinant cell produced by the aforementioned method, to cause the recombinant cell to produce 1,4-butanediol from the C1 compound.

The aspect relates to a method for producing 1,4-butanediol. In the aspect, by bringing at least one C1 compound selected from the group consisting of carbon monoxide, carbon dioxide, formic acid, and methanol into contact with the aforementioned recombinant cell, the recombinant cell is caused to produce 1,4-butanediol from the C1 compound. According to the aspect, it is possible to produce 1,4-butanediol from syngas containing carbon monoxide and/or carbon dioxide, formic acid, methanol, or the like.

Preferably, the method includes: culturing the recombinant cell by using at least one C1 compound selected from the group consisting of carbon monoxide, carbon dioxide, formic acid, and methanol as a carbon source; and obtaining 1,4-butanediol from a cultured product of the recombinant cell.

Preferably, the method includes providing a gas mainly containing carbon monoxide and hydrogen, a gas mainly containing carbon dioxide and hydrogen, or a gas mainly containing carbon monoxide, carbon dioxide and hydrogen with the recombinant cell.

The wording “provide the recombinant cell with a gas” means giving to the recombinant cell a gas as a carbon source or the like, or bringing the gas into contact with the recombinant cell.

Preferably, the recombinant cell is a Clostridium bacterium, a Moorella bacterium, or an Acetobacterium bacterium.

Another aspect of the present invention is a method for producing 1,4-butanediol, including: bringing lignocellulose, cellulose, or soluble carbohydrate in contact with the aforementioned recombinant cell or the recombinant cell produced by the aforementioned method, to cause the recombinant cell to produce 1,4-butanediol from the lignocellulose, cellulose, or soluble carbohydrate.

In the aspect, by bringing lignocellulose, cellulose, or soluble carbohydrate into contact with the aforementioned recombinant cell, the recombinant cell is caused to produce 1,4-butanediol. According to the aspect, it is possible to produce 1,4-butanediol from lignocellulose, cellulose, or soluble carbohydrate.

Preferably, the method includes: culturing the recombinant cell by using lignocellulose, cellulose, or soluble carbohydrate as a carbon source; and obtaining 1,4-butanediol from a cultured product of the recombinant cell.

Preferably, the recombinant cell is Clostridium thermocellum, Clostridium cellulolyticum, or Clostridium cellulovorans.

### Advantageous Effect of Invention

According to the recombinant cell of the present invention, it is possible to produce 1,4-butanediol from carbon monoxide, carbon dioxide, formic acid, or methanol. For example, it is possible to produce 1,4-butanediol from syngas containing carbon monoxide and/or carbon dioxide. Furthermore, it is possible to produce 1,4-butanediol from cellulose.

According to the method for producing a recombinant cell of the present invention, it is possible to produce a recombinant cell that produces 1,4-butanediol from carbon monoxide, carbon dioxide, formic acid, or methanol. Furthermore, it is possible to produce a recombinant cell that produces 1,4-butanediol from cellulose.

Similarly, according to the method for producing 1,4-butanediol of the present invention, it is possible to produce 1,4-butanediol from carbon monoxide, carbon dioxide, formic acid, or methanol. Furthermore, it is possible to produce 1,4-butanediol from cellulose.

### BRIEF DESCRIPTION OF DRAWINGS

FIG. 1 is an explanatory view showing a metabolic pathway from succinate or α-ketoglutarate to 1,4-butanediol.

FIG. 2 is an explanatory view showing an acetyl-CoA pathway and a methanol pathway.

FIG. 3 is an explanatory view showing a structure of vector pGn15_SUC(op).

FIG. 4 is an explanatory view showing a structure of vector pGn15_AKG(op).

FIG. 5 is an explanatory view showing a structure of vector pM9_SUC(op).

FIG. 6 is an explanatory view showing a structure of vector pM9_AKG.

### DESCRIPTION OF EMBODIMENT

Hereinafter, embodiments of the present invention will be described. In the present invention, the term “gene” can be replaced by the term “nucleic acid” or “DNA”.

The recombinant cell of the present invention is a recombinant cell that is acetogenic and obligatory anaerobic, wherein the recombinant cell includes a gene encoding the group of enzyme acting in a biosynthesis pathway from succinate or α-ketoglutarate to 1,4-butanediol. For example, the recombinant cell of the present invention is prepared by introducing the gene into a host cell that is acetogenic and obligatory anaerobic.

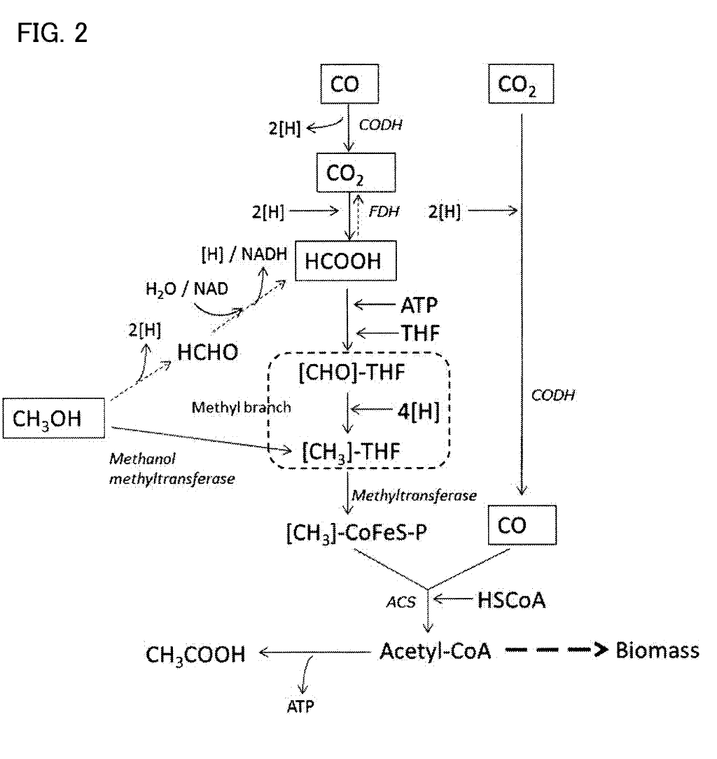

In an embodiment (first embodiment), the recombinant cell has a “function of synthesizing acetyl-CoA from methyltetrahydrofolate, carbon monoxide, and CoA”. For example, the recombinant cell is prepared by introducing the gene into a host cell having the function. Then, the recombinant cell produces 1,4-butandiol from at least one C1 compound selected from the group consisting of carbon monoxide, carbon dioxide, formic acid, and methanol. The pathway of synthesizing acetyl-CoA from methyltetrahydrofolate ([CH₃]-THF), carbon monoxide (CO), and CoA is included in, for example, acetyl-CoA pathway (Wood-Ljungdahl pathway) and methanol pathway shown in FIG. 2.

As shown in FIG. 2, in the acetyl-CoA pathway, carbon dioxide (CO₂) is reduced to carbon monoxide (CO) and a source of methyl cation in two pathways, separately. Then, by using these two carbon sources as substrate, thiol group of CoA (represented as HSCoA in FIG. 2) is acetylated to synthesize a single molecule of acetyl-CoA. Enzymes that act in the acetyl-CoA pathway include enzymes such as acetyl-CoA synthase (ACS), methyltransferase, carbon monoxide dehydrogenase (CODH), and formate dehydrogenase (FDH). It should be noted that the pathway to [CH₃]-THF from formyltetrahydrofolate ([CHO]-THF) is referred to as methyl branch.

On the other hand, the methanol pathway includes a pathway of converting methanol to formaldehyde (HCHO), and then to formic acid (HCOOH), and a pathway of deriving [CH₃]-THF from methanol.

Therefore, the pathway of synthesizing acetyl-CoA from methyltetrahydrofolate ([CH₃]-THF), carbon monoxide (CO), and CoA is common for acetyl-CoA pathway and methanol pathway.

In this embodiment, it is preferable that the recombinant cell has carbon monoxide dehydrogenase (CODH). Specifically, a cell that grows by a function of generating carbon dioxide and proton from carbon monoxide and water mainly by carbon monoxide metabolism, namely, by the action of carbon monoxide dehydrogenase is preferred. Anaerobic microorganisms having acetyl-CoA pathway and methanol pathway have carbon monoxide dehydrogenase (CODH).

Representative examples of the anaerobic microorganisms include Clostridium bacteria, Moorella bacteria, or Acetobacterium bacteria such as Clostridium ljungdahlii, Clostridium autoethanogenum, Clostridium carboxidivorans, Clostridium ragsdalei (Kopke M. et al., Appl. Environ. Microbiol. 2011, 77(15), 5467-5475), Moorella thermoacetica (same as Clostridium thermoaceticum) (Pierce E G. et al., Environ. Microbiol. 2008, 10, 2550-2573), and Acetobacterium woodii (Dilling S. et al., Appl. Environ. Microbiol. 2007, 73(11), 3630-3636). In particular, Clostridium bacteria are preferred as the host cell in the present invention because their host-vector systems and culture methods have been established.

The five species of Clostridium bacteria, Moorella bacteria, or Acetobacterium bacteria recited above are known as representative examples of syngas utilizing microorganisms.

Besides Clostridium bacteria, Moorella bacteria and Acetobacterium bacteria, Carboxydocella sporoducens sp. Nov. (Slepova T V. et al., Inter. J. Sys. Evol. Microbiol. 2006, 56, 797-800), Rhodopseudomonas gelatinosa (Uffen R L, J. Bacteriol. 1983, 155(3), 956-965), Eubacterium limosum (Roh H. et al., J. Bacteriol. 2011, 193(1), 307-308), Butyribacterium methylotrophicum (Lynd, L H. et al., J. Bacteriol. 1983, 153(3), 1415-1423) and the like may be used as a host cell on which the recombinant cell of the embodiment is based.

All of proliferation and CODH activity of the bacteria as described above are oxygen sensitive. However, oxygen insensitive CODH is also known. For example, oxygen insensitive CODH exists in other bacterial species represented by Oligotropha carboxidovorans (Schubel, U. et al., J. Bacteriol., 1995, 2197-2203), and Bradyrhizobium japonicum (Lorite M J. et al., Appl. Environ. Microbiol., 2000, 66(5), 1871-1876) (King G M et al., Appl. Environ. Microbiol. 2003, 69 (12), 7257-7265). Also in Ralsotonia bacteria which are aerobic hydrogen oxidizing bacteria, oxygen insensitive CODH exists (NCBI Gene ID: 4249199, 8019399).

As described above, there widely exist bacteria having CODH. The host cell for use in the present invention can be appropriately selected therefrom. For example, using a selective medium containing CO, CO/H₂ (gas mainly containing CO and H₂), or CO/CO₂/H₂ (gas mainly containing CO, CO₂ and H₂) as the sole carbon source and energy source, a bacterium having CODH that is usable as the host cell can be isolated in anaerobic, microaerobic or aerobic conditions.

In another embodiment (second embodiment), the recombinant cell has cellulose assimilating ability. Then, the recombinant cell produces 1,4-butandiol from lignocellulose, cellulose, or soluble carbohydrate.

Some obligatory anaerobic, acetogenic microbes can use cellulose as a carbon source. Specifically, Clostridium bacteria can effectively digest cellulose and assimilating it (Giallo J., et al., Applied Environ. Microbiol. 1985, 49(5), 1216). These microbes have cellulosome which is a protein complex with activity to effectively digest cellulose, and can effectively assimilate cellulose. Cellulosome has a structure in which many kinds of polysaccharide-degrading enzymes assemble on a scaffold protein. These polysaccharide-degrading enzymes cooperatively digest cellulose which has structural diversity. Clostridium bacteria are known as a cell producing cellulosome. Specifically, there are Clostridium cellulolyticum, Clostridium cellulosolvens, Clostridium acetobutylicum, Clostridium cellulovorans, Clostridium clariflavum, Clostridium josui, Clostridium thermocellum, Clostridium papyrosolvens, and the like. Acetivibrio cellulolyticus, Bacteroides cellulosolvens, Ruminococcus albus, Ruminococcus flavefaciens, and the like also produce cellulosome other than Clostridium bacteria.

Cellulose is converted into acetyl-CoA mainly in Embden-Meyerhof-Parnas pathway.

Specifically, the pathway consists of: 1) a pathway in which cellulose is decomposed into a saccharide such as cellobiose being a disaccharide, 2) a pathway in which a cell takes saccharide and decomposes it into glucose to synthesize pyruvate in Embden-Meyerhof-Parnas pathway, and 3) a pathway in which acetyl-CoA is synthesized from pyruvate. Then, acetyl-CoA enters citric acid cycle to synthesize succinate and α-ketoglutarate which lead to biosynthesis of 1,4-butanediol.

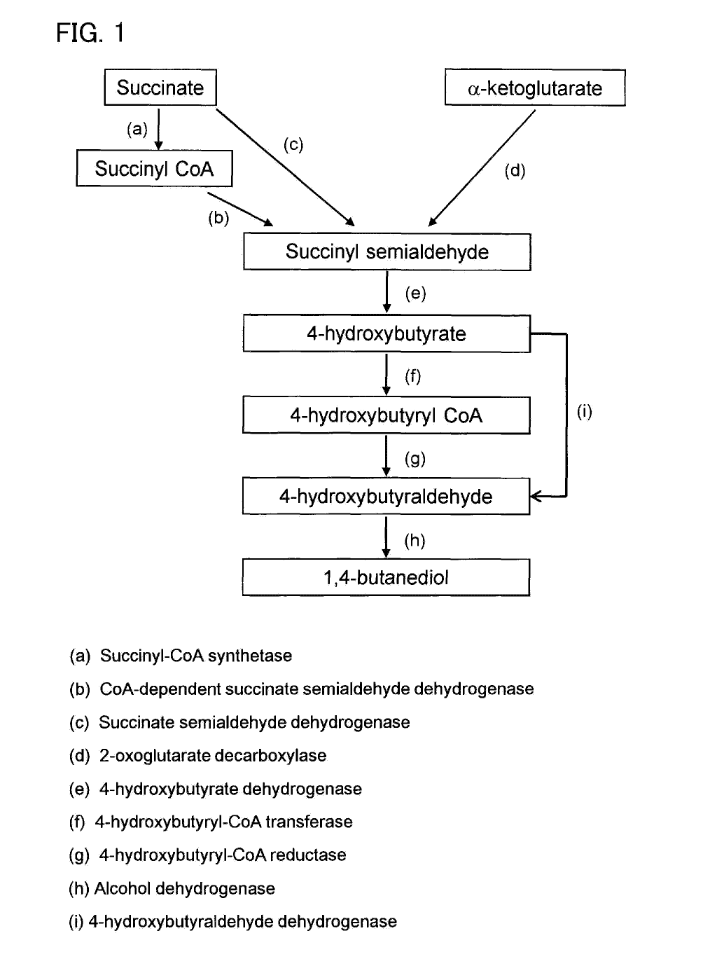

A recombinant cell of the present invention has a gene encoding a group of enzymes acting in the biosynthesis pathway from succinate or α-ketoglutarate to 1,4-butanediol (hereinafter, collectively called “1,4-butanediol biosynthesis related enzyme(s)”). For example, the gene is introduced into a host cell. The enzymes represented by (a) to (i) of FIG. 1 correspond to the 1,4-butanediol biosynthesis related enzymes.

In one aspect, a recombinant cell has a gene encoding at least one enzyme selected from the group consisting of (c) succinate semialdehyde dehydrogenase, (a) succinyl-CoA synthase, (b) CoA-dependent succinate semialdehyde dehydrogenase, (e) 4-hydroxybutyrate dehydrogenase, (f) 4-hydroxybutyryl-CoA transferase, (g) 4-hydroxybutyryl-CoA reductase, (i) 4-hydroxybutyraldehyde dehydrogenase, and (h) alcohol dehydrogenase as a group of enzymes acting in the biosynthesis pathway from succinate to 1,4-butanediol. For example, a desired recombinant cell can be obtained by introducing into a host cell a gene encoding one or more enzymes selected from the group of enzymes.

In another aspect, a recombinant cell has a gene encoding at least one enzyme selected from the group consisting of (d) 2-oxoglutarate decarboxylase, (e) 4-hydroxybutyrate dehydrogenase, (f) 4-hydroxybutyryl-CoA transferase, (g) 4-hydroxybutyryl-CoA reductase, (i) 4-hydroxybutyraldehyde dehydrogenase, and (h) alcohol dehydrogenase as a group of enzymes acting in the biosynthesis pathway from ca-ketoglutarate to 1,4-butanediol. For example, a desired recombinant cell can be obtained by introducing into a host cell a gene encoding one or more enzymes selected from the group of enzymes.

These enzymes (1,4-butanediol biosynthesis related enzymes) are not particularly limited as far as they can exert the enzymatic activity in the recombinant cell. Similarly, the genes encoding these enzymes are not particularly limited as far as they are normally transcribed and translated in the recombinant cell.

In a preferable embodiment, the 1,4-butanediol biosynthesis related enzymes are derived from bacteria. In a more preferable embodiment, the 1,4-butanediol biosynthesis related enzymes are derived from the host cell. With such a configuration, when the host cell is a bacterium, since the composition of codons used in the host cell becomes close to that of bacteria, the introduced enzyme gene can be transcribed and translated in the host cell efficiently.

Concrete examples of 1,4-butanediol biosynthesis related enzymes and genes thereof include those disclosed in Patent Document 1. For example, the following enzymes can be recited. When the host cell originally has the enzyme shown below, a gene of enzyme having higher substrate specificity, molecular activity and stability, namely having a higher value of Kcat/Km can be introduced. In this case, the enzyme gene includes a gene encoding a modified enzyme of the enzyme that is inherent in the host cell.

(c) Succinate Semialdehyde Dehydrogenase (e.g., EC 1.2.1.16, EC 1.2.1.24)

Examples of genes include (each indicated by UniProtKB No.) P76149 (E. coli); P25526 (E. coli); P94428 (Bacillus subtilis); Q55585 (Synechocystis sp.); and P38067 (Saccharomyces cerevisiae).

(a) Succinyl-CoA Synthase (Succinyl-CoA Synthetase, Succinyl-CoA Ligase: e.g., EC 6.2.1.4, EC 6.2.1.5 Etc.)

Examples of genes include (each indicated by UniProtKB/Swiss-Prot No.) POAGE9 (E. coli); POA836 (E. coli); P53598 (Saccharomyces cerevisiae); P53312 (Saccharomyces cerevisiae); P09143 (Thermus thermophilus); and 082662 (Arabidopsis thaliana). The present enzymatic activity is possessed by every organism, however, it is also effective to introduce the enzymatic activity as a foreign gene as is necessary.

(b) CoA-Dependent Succinate Semialdehyde Dehydrogenase (e.g., EC 1.2.1.79)

Examples of genes include (each indicated by UniProtKB/Swiss-Prot No.) P38947 (Clostridium kluyveri); A4YGN0 (Metallosphaera sedula) etc.

(e) 4-Hydroxybutyrate Dehydrogenase (e.g., EC 1.1.1.61)

Examples of genes include (each indicated by UniProtKB/Swiss-Prot No.) D8GUP1 (Clostridium ljungdahlii); C9YNR6 (Clostridium difficile); Q97IR6 (Clostridium acetobutylicum); Q8XYI7 (Ralstonia solanacearum); Q7MWD4 (Porphyromonas gingivalis) etc.

(f) 4-Hydroxybutyryl-CoA Transferase (e.g., EC2.8.3.a)

Examples of genes include (each indicated by UniProtKB/Swiss-Prot No.) Q9RM86 (Clostridium aminobutyricum); P38942 (Clostridium kluyveri); Q185L2 (Clostridium difficile); Q3ACH6 (Carboxydothermus hydrogenoformans); C4Z8H6 (Eubacterium rectale); I8UF15 (Porphyromonas gingivalis) etc.

(g) 4-Hydroxybutyryl-CoA Reductase (e.g., EC1.2.1.10 Etc., 4-Hydroxybutyryl-CoA Reductase Activity Shows the Catalytic Activity of the Reverse Reaction of CoA-Acylating Aldehyde Dehydrogenase)

Examples of genes include Q716S8 (Clostridium beijerinckii); Q7X4B7 (Clostridium saccharoperbutylacetonicum); A5HYN9 (Clostridium botulinum); POA9Q7 (E. coli) (these are indicated by UniProtKB/Swiss-Prot No.); GenBank CAQ57983 (Clostridium saccharobutylicum); NCBI ZP_03705305 (Clostridium methylpentosum); NCBI ZP_08533507 (Caldalkalibacillus thermarum) etc.

(h) Alcohol Dehydrogenase (e.g., EC 1.1.1.1, EC 1.1.1.2 Etc.)

Examples of genes include (each indicated by UniProtKB/Swiss-Prot No.) POA4X1 (Mycobacterium bovis); P00331 (Saccharomyces cerevisiae); P00330 (Saccharomyces cerevisiae); Q9HIM3 (Thermoplasma acidophilum); B9WPR7 (Arthrobacter sp.); P00334 (Drosophila melanogaster) etc.

The enzymatic reaction step of the two stages of (g) and (h) can be catalyzed also by the action of aldehyde/alcohol dehydrogenase (adhE: EC1.1.1.1, 1.1.1.10 etc.). That is, adhE corresponds to both the above (g) and (h). Examples of adhE include D8GU53 (Clostridium ljungdahlii), D8GU52 (Clostridium ljungdahlii), Q9ANR5 (Clostridium acetobutylicum), P0A9Q7 (E. coli), and F7TVB7 (Brevibacillus laterosporus) (each indicated by UniProtKB/Swiss-Prot No.)

(i) 4-Hydroxybutyraldehyde Dehydrogenase

This enzyme is able to catalyze conversion from 4-hydroxybutyrate to 4-hydroxybutyraldehyde reversibly, and belongs to aldehyde dehydrogenase according to the enzymatic classification (e.g., EC 1.2.1.3, EC 1.2.1.4, EC 1.2.1.5 etc.)

Examples of genes of aldehyde dehydrogenase showing 4-hydroxybutyraldehyde dehydrogenase activity include (each indicated by UniProtKB/Swiss-Prot No.) E4R8S4 (Pseudomonas putida); P23883 (E. coli); P12693 (Pseudomonas putida); P40047 (Saccharomyces cerevisiae); P25553 (E. coli); POC6D7 (Vibrio sp.); P47771 (Saccharomyces cerevisiae); G3XYI2 (Aspergillus niger) etc.

(d) 2-Oxoglutarate Decarboxylase (e.g., EC 4.1.1.71)

Examples of genes include (each indicated by UniProtKB/Swiss-Prot No.) A0R2B1 (Mycobacterium smegmatis); IOWZ48 (Rhodococcus imtechensis); G2EJR8 (Corynebacterium glutamicum); J1S9U2 (Streptomyces auratus); J7LQH4 (Arthrobacter sp.) etc.

The kind (number) of genes of “1,4-butanediol biosynthesis related enzymes” to be introduced may be at least one as far as the recombinant cell is “capable of producing 1,4-butanediol from at least one C1 compound selected from the group consisting of carbon monoxide, carbon dioxide, formic acid, and methanol”. However, when the activity of each enzyme is enhanced by introducing two or more kinds of genes, improvement in productivity of 1,4-butanediol is expected. Basically, regarding a 1,4-butanediol biosynthesis related enzyme not possessed by the host cell, a gene encoding the enzyme is introduced externally. When the enzyme is possessed by the host cell, but its molecular activity is low, it is preferred to introduce a gene encoding an enzyme having higher molecular activity.

The 1,4-butanediol biosynthesis related enzymes may be naturally occurring enzymes or enzymes modified therefrom. For example, amino acid substitution variants of each enzyme, and polypeptides that are partial fragments of each enzyme and have equivalent enzyme activity are also applicable.

In a recombinant cell of the present invention, it is preferable that ethanol generating ability of the recombinant cell is down-regulated compared to its original host cell. Furthermore, in the recombinant cell of the present invention, it is preferable that 2, 3-butanediol generating ability is down-regulated comparing to its original host cell. Many of host cells described above originally produce alcohols such as ethanol and 2, 3-butanediol. Therefore, it became able to enhance 1, 4-butandiol production by down-regulating the expression of these synthesis genes. The down-regulation includes lack of targeted gene, mutation of promoter, and mutation of enzyme genes.

In the recombinant cell of the present invention, other gene may further be introduced in addition to the gene encoding 1,4-butanediol biosynthesis related enzyme or the like. In the first embodiment, examples of the genes that can be introduced include a gene encoding an enzyme acting in acetyl-CoA pathway and methanol pathway such as acetyl-CoA synthase (ACS), methyltransferase, carbon monoxide dehydrogenase (CODH), and formate dehydrogenase (FDH). By introducing these genes, enhancement of acetyl-CoA synthesis can be expected.

In the second embodiment, examples of the genes include a gene encoding proteins which are components of cellulosome.

Similar effects are also obtained by up-regulating the expression of the genes. Herein, the up-regulation means an increase in activity by increasing the copy number of the target DNA, mutation in promoter, mutation of enzyme genes, and the like.

The technique for introducing a gene into the host cell is not particularly limited, and can be appropriately selected depending on the kind of the host cell and the like. For example, a vector that can be introduced into the host cell and can allow expression of the gene incorporated therein may be used.

For example, when the host cell is a prokaryote such as a bacterium, a vector that can self duplicate or can be incorporated in chromosome in the host cell, and contains a promoter at the position allowing transcription of the inserted gene may be used. For example, it is preferred to construct in the host cell a series of structures including a promoter, a ribosome binding sequence, the above gene and a transcription termination sequence by using the vector.

In the case where the host cell is a Clostridium bacterium (including related species such as Moorella bacteria), a shuttle vector pIMP1 between Clostridium bacterium and Escherichia coli (Mermelstein L D et al., Bio/technology 1992, 10, 190-195) may be used. The shuttle vector is a fusion vector of pUC9 (ATCC 37252) and pIM13 isolated from Bacillus subtilis (Projan S J et al., J. Bacteriol. 1987, 169 (11), 5131-5139) and is retained stably in the Clostridium bacterium. Other examples of a shuttle vector between Clostridium bacterium and Escherichia coli include pSOS95 (GenBank: AY187686.1).

For gene introduction into the Clostridium bacterium, an electroporation method is generally used. However, the introduced exogenous plasmid immediately after gene introduction is liable to be decomposed by a restriction enzyme Cac824I and the like, and is therefore very instable. For this reason, it is preferred to once amplify the vector from pIMP1 in Escherichia coli, for example, strain ER2275 having pAN1 (Mermelstein L D et al., Apply. Environ. Microbiol. 1993, 59(4), 1077-1081) carrying a methyl transferase gene from Bacillus subtilis phage 13T1, followed by a methylation treatment, and to recover the resultant vector from Escherichia coli for use in transformation by electroporation. Recently, Cac824I gene-deficient Clostridium acetobutylicum has been developed, and make it possible to stably carry a vector which is not subjected to a methylation treatment (Dong H. et al., PLoS ONE 2010, 5 (2), e9038). Furthermore, it should be noted that an unmethylated vector is efficiently introduced by amplifying the vector with NEB express which is an improved version of E. coli BL21 strain (Leang C. et al., Appl Environ Microbiol. 2013 79(4), 1102-9).

Examples of the promoter for heterologous gene expression in Clostridium bacteria include thl (thiolase) promoter (Perret S et al., J Bacteriol. 2004, 186(1), 253-257), Dha (glycerol dehydratase) promoter (Raynaud C. et al., PNAS 2003, 100(9), 5010-5015), ptb (phosphotransbutyrylase) promoter (Desai R P et al., Appl. Environ. Microbiol. 1999, 65(3), 936-945), and adc (acetoacetate decarboxylase) promoter (Lee J et al., Appl. Environ. Microbiol. 2012, 78 (5), 1416-1423). However, in the present invention, sequences of promoter regions used in operons of various metabolic systems found in the host cell or the like may be used without limited to the above examples.

For introducing plural kinds of genes into the host cell by using a vector, the genes may be incorporated in one vector, or incorporated in individual vectors. When plural kinds of genes are incorporated in one vector, these genes may be expressed under a common promoter for these genes, or expressed under individual promoters. As an exemplary form of introducing plural kinds of genes, a mode of introducing a gene encoding an enzyme acting in acetyl-CoA pathway or methanol pathway in addition to “gene encoding 1,4-butanediol biosynthesis related enzymes” can be recited.

As described above, while the known vectors that can be used in the present invention have been shown, the region involved in transcription control and replication regions such as promoter and terminator can be modified depending on the purpose. The modification includes change to other natural gene sequence in each host cell or its related species, and change to an artificial gene sequence.

By increasing the expression amount of the introduced gene in the host cell, the 1,4-butanediol discharging function and the 1,4-butanediol tolerance of the host cell by combining a variation technique such as mutation or genome shuffling in addition to the modification by the gene introduction as described above, it is possible to further improve the productivity of 1,4-butanediol.

That is, in the present invention, an exogenous gene may be incorporated in a genome of the host cell or incorporated in a plasmid.

In one preferred embodiment, the recombinant cell is tolerant to at least 400 mM 1,4-butanediol. With such a configuration, it is possible to mass-produce 1,4-butanediol. For example, the recombinant cell having such characteristics can be obtained by subjecting host cells to an appropriate variation treatment to select a host cell having the intended characteristics, and using the host cell.

One aspect of the method for producing 1,4-butanediol of the present invention includes bringing at least one C1 compound selected from the group consisting of carbon monoxide, carbon dioxide, formic acid, and methanol into contact with the aforementioned recombinant cell to cause the recombinant cell to produce 1,4-butanediol from the C1 compound.

Preferably, the method includes: culturing the recombinant cell by using at least one C1 compound selected from the group consisting of carbon monoxide, carbon dioxide, formic acid, and methanol as a carbon source; and obtaining 1,4-butanediol from a cultured product of the recombinant cell. The C1 compound used as a carbon source may be used singly or in combination of two or more. The C1 compound is preferably used as a main carbon source, and more preferably as the sole carbon source.

Also, it is preferred to provide hydrogen (H₂) concurrently as an energy source.

The method for culturing the recombinant cell of the present invention is not particularly limited, and may be appropriately conducted depending on the kind of the host cell and the like. When the recombinant cell is a Clostridium bacterium (obligatory anaerobic, strictly anaerobic), it is cultured, for example, in a nutrient condition including inorganic salts required for growth, and syngas. Preferably, it is cultured under a pressurized condition at about 0.2 to 0.3 MPa (absolute pressure). Further, for improving initial proliferation and attained cell density, small amounts of organic substances such as vitamin, yeast extract, corn steep liquor, and Bacto Tryptone may be added.

When the recombinant cell is aerobic or facultative anaerobic, for example, it may be cultured in a liquid medium under aeration and stirring.

It is also possible to produce 1,4-butanediol without culturing the recombinant cell. That is, regardless of whether cell division (cell proliferation) is involved or not, it is possible to produce 1,4-butanediol by bringing the C1 compound into contact with the recombinant cell. For example, it is possible to continuously produce 1,4-butanediol by continuously supplying a fixed recombinant cell with the C1 compound. Also in the present aspect, regarding these C1 compounds, only one C1 compound may be used, or a combination of two or more C1 compounds may be used. Also, it is preferred to provide hydrogen (H₂) concurrently as an energy source.

In a preferred embodiment, the recombinant cell is provided with a gas mainly containing carbon monoxide and hydrogen (CO/H₂), a gas mainly containing carbon dioxide and hydrogen (CO₂/H₂), or a gas mainly containing carbon monoxide, carbon dioxide and hydrogen (CO/CO₂/H₂). In other words, 1,4-butanediol is produced from carbon monoxide or carbon dioxide in such a gas by culturing the recombinant cell by using the gas as a carbon source, or by bringing the gas into contact with the recombinant cell. Also in this case, hydrogen is used as an energy source.

In another preferred embodiment, the recombinant cell is provided with methanol/carbon monoxide, methanol/carbon dioxide, or methanol/carbon monoxide/carbon dioxide. In other words, 1,4-butanediol is produced from methanol, carbon monoxide, or carbon dioxide by culturing the recombinant cell by using methanol and the gases as a carbon source, or by bringing methanol and the gases into contact with the recombinant cell. Modes for providing the recombinant cell with methanol and the gases include providing methanol and the gases concurrently.

Furthermore, 1,4-butanediol may be produced from formic acid and/or methanol by providing the recombinant cell with formic acid and/or methanol. In other words, 1,4-butanediol can also be produced from formic acid and/or methanol by culturing the recombinant cell using, as a carbon source, formic acid or methanol solely or in addition to carbon monoxide and/or carbon dioxide, or by bringing formic acid and/or methanol into contact with the recombinant cell.

A method for producing 1,4-butanediol of the present invention includes bringing lignocellulose, cellulose, or soluble carbohydrate in contact with the aforementioned recombinant cell to cause the recombinant cell to produce 1,4-butanediol from the lignocellulose, cellulose, or soluble carbohydrate. Preferably, the method includes culturing the recombinant cell by using lignocellulose, cellulose, or soluble carbohydrate as a carbon source, and obtaining 1,4-butanediol from a cultured product of the recombinant cell.

Cellulose substrates include, for example cellulose biomass. Examples of the cellulose biomass includes rice straw, vegetable scraps, bagasse, wood pulp, wood chips, sawdust, used papers, rayon, cotton, hemp, thread, corrugated cardboards, and crystalline cellulose. While it is possible to make these cellulose substrates directly contact with the recombinant cell of this invention, it is preferable that these substrates are treated physically, chemically, and/or enzymatically, in advance of contact with the cells, or in the same time as culture of the cell. As a physical treatment, for example, ball mill, vibrating mill, pressurized hot water, cooking blasting, and the like are available. As a chemically treatment, acids, alkalis, and the like are available. As an enzymatic treatment, hemicellulases, cellulases, liquids of these enzymes, and the like are available.

The produced 1,4-butanediol is accumulated in the cell or released outside the cell. For example, purified 1,4-butanediol can be obtained by using the recombinant cell prepared from a host cell of a Clostridium bacterium or a Moorella bacterium, and recovering 1,4-butanediol released outside the cell, followed by isolation and purification.

Herein, combinations of carbon monoxide, carbon dioxide, formic acid, and methanol that can be provided to the recombinant cell in the case where the host cell has the acetyl-CoA pathway and the methanol pathway (FIG. 2) are described.

In acetyl-CoA synthesis by the acetyl-CoA pathway, a synthesis process of acetyl-CoA from CoA, methyltetrahydrofolate ([CH₃]-THF), and CO by the actions of methyltransferase, Corrinoid iron-sulfur protein (CoFeS-P), acetyl-CoA synthase (ACS), and CODH is essential (Ragsdale S W et al., B.B.R.C. 2008, 1784(12), 1873-1898).

On the other hand, it is known that adding formic acid and/or methanol besides CO and/or CO₂ as a carbon source in culturing of Butyribacterium methylotrophicum increases the content of tetrahydrofolate (THF) in CO metabolism, namely, methyl branch in the acetyl-CoA pathway, and activities of CODH, formate dehydrogenase (FDH) and hydrogenase required in CO metabolism (Kerby R. et al., J. Bacteriol. 1987, 169(12), 5605-5609). Also in Eubacterium limosum or the like, it is demonstrated that high proliferation is achieved by using CO₂ and methanol as a carbon source in an anaerobic condition (Genthner B R S. et al., Appl. Environ. Microbiol., 1987, 53(3), 471-476).

The influence of methanol on syngas utilizing microorganisms, and the results of genome analysis of Moorella thermoacetica (Clostridium thermoaceticum), Clostridium jungdahlii and the like (Pierce E. et al., Environ. Microbiol. 2008, 10(10), 2550-2573; Durre P. et al., PNAS 2010, 107(29), 13087-13092) can give an explanation for involvement of the methanol pathway as shown in FIG. 2 as a donor of a methyl group in the acetyl-CoA pathway (Wood-Ljungdahl pathway) in these microorganism species.

Actually in some Clostridium bacteria, the forward activity of formate dehydrogenase (FDH) (EC 1.2.1.2/1.2.1.43: Formate+NAD(P)⁺⇔CO₂+NAD(P)H) (formation of CO₂ from formate) is confirmed (Liu C L et al., J. Bacteriol. 1984, 159(1), 375-380; Keamy J J et al., J. Bacteriol. 1972, 109(1), 152-161). Therefore, in these strains, a reaction in the direction of generating CO₂ from methanol (CH₃OH) and/or formic acid (HCOOH) can partly proceed when CO₂ and/or CO is deficient (FIG. 2). This can also be explained by the phenomenon that formate dehydrogenase activity and CODH activity increase by addition of CH₃OH (Kerby R et al., J. Bateriol. 1987, 169(12), 5605-5609) as described above. In other words, these can be proliferated with formic acid (HCOOH) or methanol (CH₃OH) as the sole carbon source.

Even if the host cell strain inherently lacks the forward activity of formate dehydrogenase, it may be provided with the forward activity by gene modification such as introduction of mutation, introduction of foreign gene, or genome shuffling.

For these reasons, it is possible to produce 1,4-butanediol using the following gas or liquid when the host cell has the acetyl-CoA pathway and the methanol pathway.
CO
CO₂
CO/H₂
CO₂/H₂
CO/CO₂/H₂
CO/HCOOH
CO₂/HCOOH
CO/CH₃OH
CO₂/CH₃OH
CO/H₂/HCOOH
CO₂/H₂/HCOOH
CO/H₂/CH₃OH
CO₂/H₂/CH₃OH
CO/CO₂/H₂/HCOOH
CO/CO₂/H₂/CH₃OH
CH₃OH/H₂
HCOOH/H₂
CH₃OH
HCOOH

When the recombinant cell of the present invention is cultured exclusively for cell proliferation, rather than for production of 1,4-butanediol, it is not necessary to use carbon monoxide and/or carbon dioxide as a carbon source. For example, the recombinant cell may be cultured using other carbon sources such as saccharides or glycerin which is easily assimilated by the recombinant cell of the present invention

In the following, the present invention will be described more specifically by way of examples. However, the present invention is not limited to these examples.

### Example 1

1,4-BDO Production by Syngas Utilizing Bacterium C. ljungdahlii (Succinate Pathway)

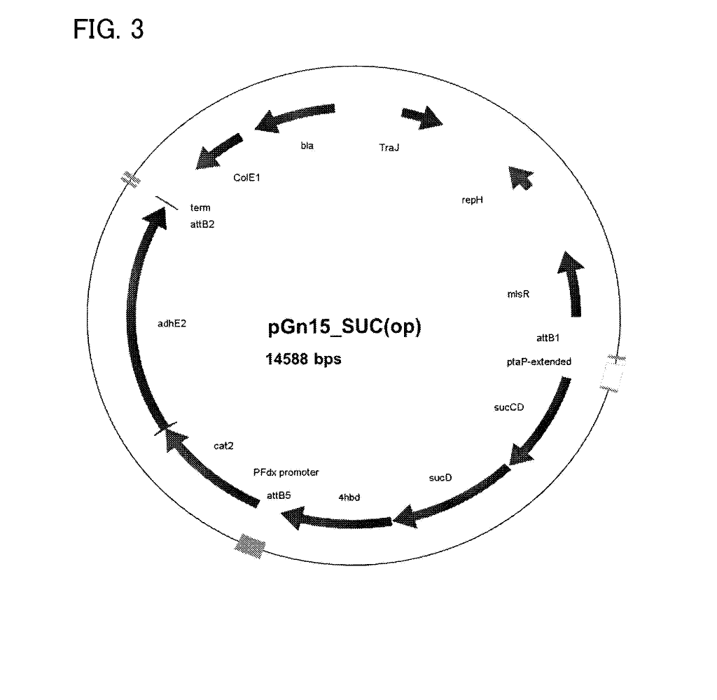

Clostridium/E. coli shuttle vector pGn15_SUC(op) (SEQ ID NO: 1, FIG. 3.) was introduced into C. ljungdahlii by electroporation (performed with a BioRad Micropulser (BioRad, Hercules, Calif., USA) at 0.63 kV) and the transformants were collected on YTF agar plates (YTF-medium containing per liter, 16 g tryptone, 10 g yeast extract, 4 g NaCl, 2 mM L-cysteine supplemented with 1% Xylose plus 1.5% agar).

For the electroporation the following protocol was used. All work was always performed under strictly anaerobic conditions and, except the centrifugation steps and the storage of the cryocultures, inside the Whitley Anaerobic Workstation A55 (Don Whitley Scientific Limited, United Kingdom) with the gas composition 10% CO₂, 5% H₂ and 85% N₂. Clostridium ljungdahlii strain (DSM No.: 13528) was ordered from DSMZ (Deutsche Sammlung von Mikroorganismen und Zellkulturen) and received as freeze dried culture. The general recommendation by DSMZ for handling strictly anaerobic microorganisms and for strain specific cultivation conditions was employed. The pellet was dissolved in 20 mL YTF-medium (1 L contains 16 g tryptone, 10 g yeast extract, 4 g NaCl, 2 mM L-cysteine supplemented with 1% Xylose) in a 50 mL centrifuge tube. The culture was taken at OD660 of around 2.0 to inoculate 20 mL fresh medium with 1% of the preculture. This was repeated three times and then 100 mL medium were inoculated the same way in a 200 mL flask for preparation of competent cells. For the destabilization of the cell wall 40 mL DL-threonine were added. The cells were harvested at OD600 of around 0.3 and then washed three times with a volume of 100 mL of ice cold washing buffer (270 mM sucrose, 1 mM MgCl₂, 20 mM BisTris, pH 5.9). The supernatant was discarded and after each washing step the samples were centrifuged at 4° C. and 4000 g for 5 min in 50 mL centrifuge tubes to spin down the cells. Finally, the washed cell pellet was dissolved in 2 mL washing buffer supplemented with 10% DMSO and 25 μL aliquots of cells were prepared in 1.5 mL tubes. The competent cells were then stored up to three months at −80° C.

The methylation of DNA is generally an important issue for the transformation of clostridia. For Clostridium ljungdahlii E. coli B-strains with Dcm⁻ Dam⁺ methylation have descripted as more efficient for plasmid preparation with subsequent electroporation (Leang et al. 2013). For the transformation of the 1,4 BDO cluster plasmid DNA was isolated from the K-strain NEB10B (New England Biolabs, Frankfurt am Main, Germany), because the plasmid stability is considered to be higher especially when pathways are driven by constitutive promoters. The successful transformation of Clostridium ljungdahlii with DNA from the E. coli Dcm⁺ Dam⁻ strain NEB10B suggests that a specific methylation is not a fundamental step for the DNA transfer into Clostridium ljungdahlii. For the DNA transfer the cells were thawed on ice and were mixed with 3 μg of the plasmid

The DNA/cell mix was then transferred to a prechilled 0.1 mm gap cuvette and electroporation was performed with a BioRad Micropulser (BioRad, Hercules, Calif., USA) at 0.63 kV Next the cells were regenerated at 37° C. in 20 mL YTF-medium for at least 12 hours. Then the antibiotic clarithromycin was added to the culture (dissolved in 70% ethanol, final concentration 5 μg/mL) and the cells were further incubated for 6 hours. Then different amounts of the cell culture were mixed with YTF-agar medium. Around 30 mL of YTF-agar medium (containing 5 μg/mL clarithromycin) at 37° C. were mixed with 1 μL, 10 μL, 100 μL, 1 mL, 2 mL, 3 mL and the rest of the 20 mL regeneration culture in petri dishes (9 cm diameter) and incubated for at least 5 days at 37° C. The obtained colonies were picked from plates, where 1-100 μL culture had been added, and only when they showed the morphology of Clostridium ljungdahlii. Fifteen milliliter centrifuge tubes were used for further cultivation and precultures. Clarithromycin was always added to final concentration of 5 μg/mL.

The vector pGn15_SUC(op) includes 1,4-BDO synthesizing gene cluster (FIG. 3) encoding sucCD from Escherichia coli, sucD from Clostridium kluyveri, 4hbd from Porphyromonas gingivalis, cat2 from Porphyromonas gingivalis, and adhE2 from Clostridium acetobutylicum. The collected transformants were named CLJU_pGn15_SUC(op).

The transformant named CLJU_pGn15_SUC(op) described above and C. ljungdahlii wildtype were anaerobically cultured in the YTF medium (containing per liter, 16 g tryptone, 10 g yeast extract, 4 g NaCl, 2 mM L-cysteine supplemented with 1% Xylose or Fructose). The culture was conducted in the 50 mL of sealed bottle with 200 mL of the above medium at 37° C. and the culture bottles were shaken. When the OD600 of the culture medium was reached 2.0, the medium was centrifuged and the supernatant was collected. The supernatant was filtered by 0.22 m filter and analyzed by GCMS (QP2010S GC-MS system (Shimadzu), InterCap FFAP (GL Sciences), mobile phase: 100% Methanol, carrier gas:helium, split ratio:1:10, injection temperature: 220° C.).

GC-MS Analysis Parameters for Operated Columns:

Column: InterCap FFAP (GL Sciences)

Dimension: 30 m×0.25 mm, 0.25 μm

Mobile phase: 100% Methanol

Carrier gas: Helium

Split ratio: 1:10

Total flow: 14.4 mL/min

Injection Temperature: 220° C.

GC-MS Analysis using InterCap FFAP for detecting 1,4-BDO

| Rate [° C./min] | Final Temperature [° C.] | Hold Time [min] |
|---|---|---|
| — | 60 | 1.00 |
| 5.0 | 70 | 0.00 |
| 35.0 | 220 | 2.00 |

As a result, while the CLJU_pGn15_SUC(op) introduced with 1,4-BDO synthesizing gene cluster produced significant amount of 1,4-BDO with the yield of 20-40 μM, the wildtype did not produce 1,4-BDO. It is shown that the syngas utilizing bacterium C. ljungdahlii introduced with 1,4-BDO synthesizing gene can produce 1,4-BDO.

### Example 2

1,4-BDO Production by Syngas Utilizing Bacterium Clostridium ljungdahlii (α-Ketoglutarate Pathway)

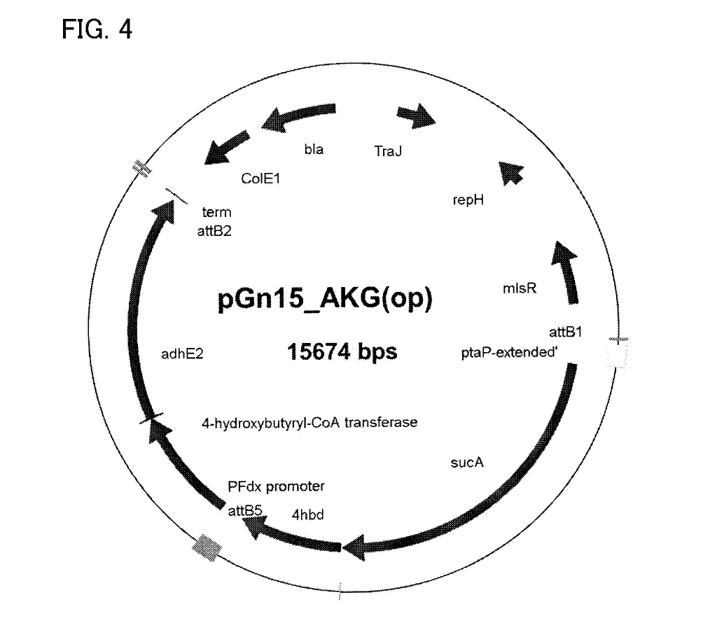

Clostridium/E. coli shuttle vector pGn15_AKG (op) (SEQ ID NO: 2, FIG. 4) was introduced into C. ljungdahlii by electroporation (performed with a BioRad Micropulser (BioRad, Hercules, Calif., USA) at 0.63 kV) and the transformants were collected on YTF agar plates (YTF-medium containing per liter, 16 g tryptone, 10 g yeast extract, 4 g NaCl, 2 mM L-cysteine supplemented with 1% Xylose plus 1.5% agar). The vector pGn15_AKG(op) includes 1,4-BDO synthesizing gene cluster (FIG. 4) encoding α-ketoglutarate decarboxylase (sucA from Mycobacterium bovis), 4-hydroxybutyrate dehydrogenase (4hbd from Porphyromonas gingivalis), 4-hydroxybutyryl CoA transferase (cat2 from Porphyromonas gingivalis), and bifunctional 4-hydroxybutyryl CoA reductase and alcohol dehydrogenase (adhE2 from Clostridium acetobutylicum). The collected transformants were named CLJU_pGn15_AKG(op).

The transformant CLJU_pGn15_AKG(op) described above and C. jungdahlii wildtype were anaerobically cultured in the ATCC medium (containing 1 g/L Yeast extract, 2 mM cysteine, pH 6.0, adapted from Koepke Dissertation, Ulm 2009). The culture was conducted in the 50 mL of sealed bottle with 200 mL of the above medium at 37° C. and the culture bottles were shaken. The head-space of the bottle was filled up with a mix Gas of CO/CO₂/H₂=(1:1:1) at the pressure of 1 MPa (absolute pressure), and then sealed with butyl gum caps and aluminum caps.

When OD600 of the culture medium was reached 2.0, the medium was centrifuged and the supernatant was collected. The supernatant was filtered by 0.22 μm filter and analyzed by GCMS (QP2010S GC-MS system (Shimadzu), InterCap FFAP (GL Sciences), mobile phase: 100% Methanol, carrier gas:helium, split ratio:1:10, injection temperature: 220° C.).

This transformant was also used as a Pre-culture for syngas fermentation. Pre-cultures were prepared in flasks containing 50 mL ATCC media (pH 6.0). Either Syngas (1/1/1; 1 bar overpressure) or Syngas (1/1/1; 1 bar overpressure) plus 1% fructose was used as carbon source in precultures. Selection was maintained with clarithromycin (10 μg/mL). Pre-cultures required at least 48 hours at 37° C. to grow before being inoculated into the fermenter. Pre-cultures growing on syngas required more than 72 hours before inoculation. For the fermentation 2 μL of ATCC media containing an additional 50 mL of 0.8 M BIS-TRIS pH 6.0 and 1.5 mL antifoam (Struktol J673) was prepared. For each fermentation-unit 500 mL of 1M NaOH as base was prepared and autoclaved separately, furthermore 200 μL of resazurin (20 mg/mL) were added before autoclaving the media. After autoclaving, the trace elements, vitamin-solution, L-Cystein and antibiotics were added and the pH control was activated so that pH 6.0 could be maintained. To deoxygenate the vessels a continuous nitrogen gasflow (0.8 L/min; 10-15 min) was used. Afterwards the media was saturated with syngas (1/1/1; 0.25 L/min) for 15 min. Samples were taken two times a day for analysis of the supernatant via GC/MS (6 mL) and every 24 h for targeted proteomics analysis via LC/MS-MS (50-100 mL depending on the OD600 which was also measured twice daily and recorded. After 5-6 days the fermentation broth was harvested and 1,4-BDO was extracted using solvent extraction. The method employed was via a dichloromethane and salt extraction which enabled the reduction and concentration of the 2 L fermentation broth down to less than 100 mL. Five hundred (500) μL of the extract was pipetted into a GCMS analysis tube, and then 1 μL thereof was injected.

As a result, while the CLJU_pGn15_AKG(op) introduced with 1,4-BDO synthesizing gene cluster produced significant amount of 1,4-BDO with the yield of 40-170 μM, the wildtype did not produce 1,4-BDO. It is shown that the syngas-utilizing bacterium C. ljungdahlii introduced with 1,4-BDO synthesizing gene can produce 1,4-BDO.

### Example 3

1,4-BDO Production by Cellulolytic Bacterium Clostridium cellulolyticum (Succinate-Pathway)

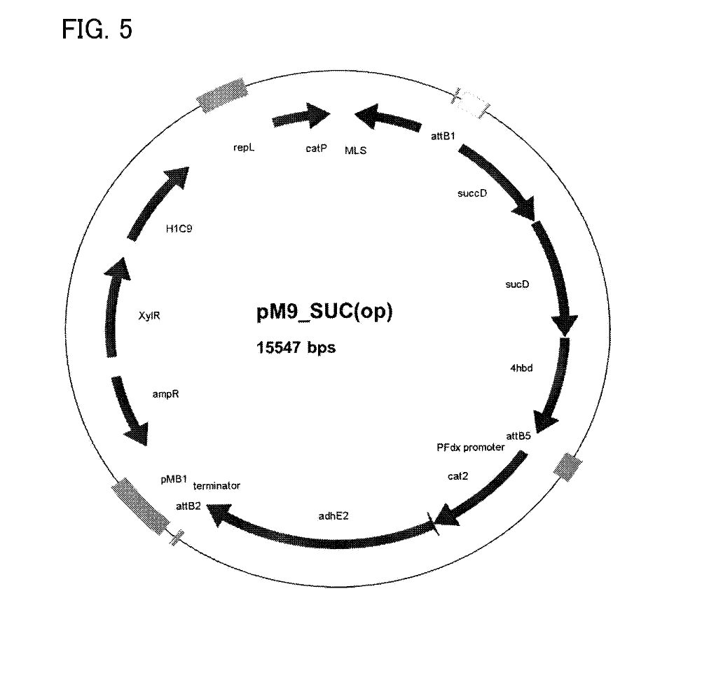

Clostridium/E. coli shuttle vector, pM9_SUC(op) (SEQ ID NO: 3, FIG. 5) was introduced into C. cellulolyticum by electroporation (at 1.6 kV) and transformants were collected on CM3 agar plates (1.3 g L⁻¹ (NH₄)₂ SO₄, 1.5 g L⁻¹ KH₂PO₄, 2.9 g L⁻¹ K₂HPO₄×3 H₂O, 0.2 g L⁻¹ MgCl₂×6 H₂O, 75.0 mg L⁻¹ CaCl₂×2 H₂O, 1.25 mg L⁻¹ FeSO₄×7 H₂O, 1.0 mL L⁻¹ trace element solution SL-10, 1.0 mg L⁻¹ Resazurin, 2.0 g L⁻¹ yeast extract, 2.5 g L⁻¹ Na₂CO₃, 0.5 g L⁻¹ L-cysteine-HCL×H₂O, 6.0 g L⁻¹ D-cellobiose and 15.0 g L⁻¹ agar). The vector pM9_SUC(op) includes 1,4-BDO synthesizing gene cluster (FIG. 5) encoding succinyl CoA synthetase (sucCD from Escherichia coli), CoA dependent succinate semialdedhyde dehydrogenase (sucD from Clostridium kluyveri), 4hbd from Porphyromonas gingivalis, cat2 from Porphyromonas gingivalis, and adhE2 from Clostridium acetobutylicum. The collected transformants were named CCE_pM9_SUC(op).

The CCE_pM9_SUC(op) and CCE wildtype described above were anaerobically cultured in the CM3 medium (CM3 agar without agar). The culture was conducted in the 50 mL of sealed bottle with 200 mL of the above medium at 34° C. The culture bottles were sealed with butyl gum caps and aluminum caps.

When OD600 of the culture medium was reached 2.0, the medium was centrifuged and the supernatant was collected. The supernatant was filtered by 0.22 μm filter and analyzed by GCMS (QP2010S GC-MS system (Shimadzu), InterCap FFAP (GL Sciences), mobile phase: 100% Methanol, carrier gas:helium, split ratio:1:10, injection temperature: 220° C.).

As a result, while the pM9_SUC(op) introduced with 1,4-BDO synthesizing gene cluster produced significant amount of 1,4-BDO with the yield of 60-120 μM, the wildtype did not produce 1,4-BDO. It is shown that the cellulolytic bacterium C. cellulolyticum introduced with 1,4-BDO synthesizing gene can produce 1,4-BDO.

### Example 4

1,4-BDO Production by Cellulolytic Bacterium Clostridium cellulolyticum (α-Ketoglutarate-Pathway)

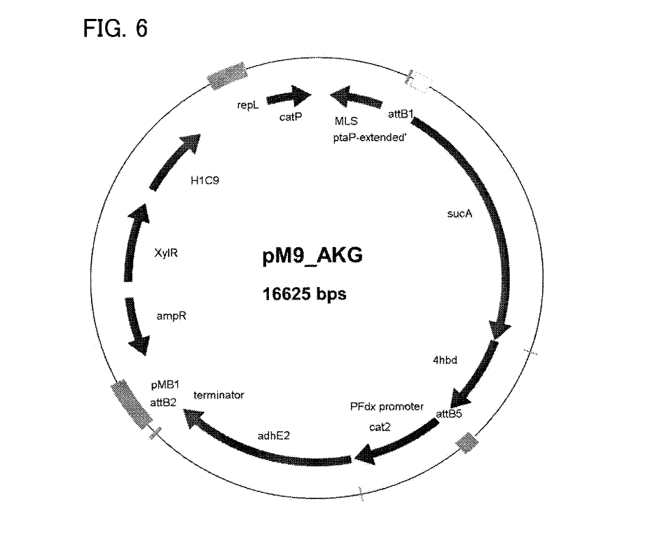

Clostridium/E. coli shuttle vector, pM9_AKG (SEQ ID NO: 4, FIG. 6) was introduced into C. cellulolyticum by electroporation (at 1.6 kV) and the transformants were collected on CM3 agar plates (containing 1.3 g L⁻¹ (NH₄)₂ SO₄, 1.5 g L⁻¹ KH₂PO₄, 2.9 g L⁻¹ K₂HPO₄×3 H₂O, 0.2 g L⁻¹ MgCl₂×6 H₂O, 75.0 mg L⁻¹ CaCl₂×2 H₂O, 1.25 mg L⁻¹ FeSO₄×7 H₂O, 1.0 mL L⁻¹ trace element solution SL-10, 1.0 mg L⁻¹ Resazurin, 2.0 g L⁻¹ yeast extract, 2.5 g L⁻¹ Na₂CO₃, 0.5 g L⁻¹ L-cysteine-HCL×H₂O, 6.0 g L⁻¹ D-cellobiose and 15.0 g L⁻¹ agar). The vector pM9_AKG includes 1,4-BDO synthesizing gene cluster (FIG. 6) encoding sucA from Mycobacterium bovis, 4hbd from Porphyromonas gingivalis, cat2 from Porphyromonas gingivalis, and adhE2 from Clostridium acetobutylicum. The collected transformants were named CCE_pM9_AKG.

The CCE_pM9_AKG and CCE wildtype described above were anaerobically cultured in the CM3 medium (CM3 agar without agar). The culture was conducted in the 50 mL of sealed bottle with 200 mL of the above medium at 34° C. The culture bottles were sealed with butyl gum caps and aluminum caps.

When the OD600 of the culture medium was reached 2.0, the medium was centrifuged and the supernatant was collected. The supernatant was filtered by 0.22 μm filter and analyzed by GCMS (QP2010S GC-MS system (Shimadzu), InterCap FFAP (GL Sciences), mobile phase: 100% Methanol, carrier gas:helium, split ratio:1:10, injection temperature: 220° C.).

As a result, while the pM9_AKG introduced with 1,4-BDO synthesizing gene cluster produced significant amount of 1,4-BDO with the yield of 60 μM, the wildtype did not produce 1,4-BDO. It is shown that the cellulolytic bacterium C. cellulolyticum introduced with 1,4-BDO synthesizing gene can produce 1,4-BDO.

### Example 5

1,4-Butanediol Production from Syngas by Clostridium ljungdahlii (Succinate-Pathway)

(1) Construction of a Vector that Expresses 1,4-Butanediol Biosynthesis Related Enzymes

The artificial gene of SEQ ID NO: 5 (6774 bp), which is a gene cluster encoding 1,4-butanediol biosynthesis related enzymes, was synthesized. The gene cluster includes sucD (CoA-deopendent succinate semialdehyde dehydrogenase; Gene ID: 5394466), 4Hb (4-hydroxybutyrate dehydrogenase; Gene ID: 2552693), abfT (4-hydroxybutyryl-CoA reductase, EMBL_CAB60036.2), adhE2 (alcohol dehydrogenase; EMBL_AAK09379.1) genes, and T7 terminator, and further includes a BamHI recognition site and a XhoI recognition site at a terminal and the other terminal of the cluster, respectively.

The artificial DNA of SEQ ID NO: 6 (120 bp) was introduced into the BamHI/NheI site of Clostridium/E. coli shuttle vector pSOS95 (Genbank: AY187686.1) to give pSOS-MS. A gene fragment obtained by digesting the artificial gene of SEQ ID NO: 5 with BglII/XhoI was introduced into the BamHI/NheI site of pSOS-MS to give pSOS-BDO. NBL express was used for the cloning procedure. Like pSOS95, pSOS-BDO also functions as a Clostridium/E. coli shuttle vector.

(2) Preparation of a Recombinant Cell that Produces 1,4-Butanediol

The pSOS-BDO prepared in item (1) was introduced by electroporation into Clostridium ljungdahlii (DSM13528/ATCC55383) to give a transformant. As a control, the pSOS-MS was also introduced into Clostridium ljungdahlii (DSM13528/ATCC55383) to give another transformant. The electroporation was conducted by the methods recommended in Appl. Environ. Microbiol. 2013, 79 (4): 1102-9.

### (3) 1,4-BDO Production by the Transformant

The two kinds of transformants obtained in item (2) were anaerobically cultured at 37° C. Fructose-free ATCC 1754 PETC medium containing 4 pg/mL clarithromycin was used for the culture. The culture was conducted in a 125 mL sealed bottle with 45 mL of the medium and a mixed gas of CO/CO₂/H₂=33/33/34 at the pressure of 0.25 MPa (absolute pressure). The bottle was sealed with butyl gum caps and aluminum caps, and then shaken. When the OD600 of the culture medium was reached 0.9, the medium was centrifuged and the supernatant was collected. The supernatant was analyzed by LCMS.

As a result, while the transformant introduced with pSOS-BDO produced 1,4-BDO, the transformant introduced with pSOS-MS did not produce 1,4-BDO. It is shown that recombinant Clostridium ljungdahlii introduced with 1,4-BDO synthesizing gene cluster can produce 1,4-BDO from syngas.

1. A recombinant cell that is acetogenic and obligatory anaerobic, wherein
the recombinant cell comprises (i) a gene encoding a group of enzymes comprising CoA-dependent succinate semialdehyde dehydrogenase, 4-hydroxybutyrate dehydrogenase, 4-hydroxybutyryl-CoA transferase, 4-hydroxybutyryl-CoA reductase, and alcohol dehydrogenase, or (ii) a gene encoding a group of enzymes comprising 2-oxoglutarate decarboxylase, 4-hydroxybutyrate dehydrogenase, 4-hydroxybutyryl-CoA transferase, 4-hydroxybutyryl-CoA reductase, and alcohol dehydrogenase,
the gene is expressed in the recombinant cell, and
the recombinant cell produces 1,4-butandiol.

2. (canceled)

3. The recombinant cell according to claim 1, wherein
the recombinant cell has a function of synthesizing acetyl-CoA from methyltetrahydrofolate, carbon monoxide, and CoA, and
the recombinant cell produces 1,4-butandiol from at least one C1 compound selected from the group consisting of carbon monoxide, carbon dioxide, formic acid, and methanol.

4. The recombinant cell according to claim 1, wherein the recombinant cell has carbon monoxide dehydrogenase.

5. The recombinant cell according to claim 1, wherein the recombinant cell is a Clostridium bacterium, a Moorella bacterium, or an Acetobacterium bacterium.

6. The recombinant cell according to claim 1, wherein
the recombinant cell has cellulose assimilating ability, and
the recombinant cell produces 1,4-butandiol from lignocellulose, cellulose, or soluble carbohydrate.

7. The recombinant cell according to claim 1, wherein the recombinant cell produces cellulosome.

8. The recombinant cell according to claim 7, wherein the recombinant cell is a Clostridium bacterium.

9. The recombinant cell according to claim 8, wherein the recombinant cell is Clostridium thermocellum, Clostridium cellulolyticum, or Clostridium cellulovorans.

10. The recombinant cell according to claim 1, wherein at least one of the genes is derived from a bacterium.

11. The recombinant cell according to claim 1, wherein at least one of the genes is derived from its original host cell.

12-13. (canceled)

14. The recombinant cell according to claim 1, wherein ethanol generating ability of the recombinant cell is down-regulated compared to that of its original host cell.

15. The recombinant cell according to claim 1, wherein 2,3-butanediol generating ability of the recombinant cell is down-regulated compared to that of its original host cell.

16. A method for producing a recombinant cell, comprising:
a first step of providing a host cell that is acetogenic and obligatory anaerobic; and
a second step of introducing a gene encoding a group of enzymes comprising CoA-dependent succinate semialdehyde dehydrogenase, 4-hydroxybutyrate dehydrogenase, 4-hydroxybutyryl-CoA transferase, 4-hydroxybutyryl-CoA reductase, and alcohol dehydrogenase, or (ii) a gene encoding a group of enzymes comprising 2-oxoglutarate decarboxylase, 4-hydroxybutyrate dehydrogenase, 4-hydroxybutyryl-CoA transferase, 4-hydroxybutyryl-CoA reductase, and alcohol dehydrogenase, into the host cell,
wherein the gene is expressed in the recombinant cell, and the recombinant cell produces 1,4-butandiol.

17-30. (canceled)

31. A method for producing 1,4-butanediol, comprising:
bringing at least one C1 compound selected from the group consisting of carbon monoxide, carbon dioxide, formic acid, and methanol into contact with the recombinant cell according to claim 1, to cause the recombinant cell to produce 1,4-butanediol from the C1 compound.

32. The method according to claim 31, comprising:
culturing the recombinant cell by using at least one C1 compound selected from the group consisting of carbon monoxide, carbon dioxide, formic acid, and methanol as a carbon source; and
obtaining 1,4-butanediol from a cultured product of the recombinant cell.

33. The method according to claim 31, comprising:
providing a gas mainly containing carbon monoxide and hydrogen, a gas mainly containing carbon dioxide and hydrogen, or a gas mainly containing carbon monoxide, carbon dioxide and hydrogen with the recombinant cell.

34. The method according to claim 31, wherein the recombinant cell is a Clostridium bacterium, a Moorella bacterium, or an Acetobacterium bacterium.

35. A method for producing 1,4-butanediol, comprising:
bringing lignocellulose, cellulose, or soluble carbohydrate in contact with the recombinant cell according to claim 1, to cause the recombinant cell to produce 1,4-butanediol from the lignocellulose, cellulose, or soluble carbohydrate.

36. The method according to claim 35, comprising:
culturing the recombinant cell by using lignocellulose, cellulose, or soluble carbohydrate as a carbon source; and
obtaining 1,4-butanediol from a cultured product of the recombinant cell.

37. The method according to claim 35, wherein the recombinant cell is Clostridium thermocellum, Clostridium cellulolyticum, or Clostridium cellulovorans.
